(19) United States
(12) Patent Application Publication
(10) Pub. No.: US 2024/0272164 A1
(43) Pub. Date: Aug. 15, 2024

(54) METHODS FOR CLASSIFYING TUMORS AND USES THEREFOR

(71) Applicant: THE UNIVERSITY OF QUEENSLAND, St. Lucia (AU)

(72) Inventors: Fiona SIMPSON, Paddington (AU); Shannon Rose LEFTWICH, Burpengary (AU)

(21) Appl. No.: 18/413,636

(22) Filed: Jan. 16, 2024

Related U.S. Application Data
(63) Continuation of application No. 16/076,792, filed on Aug. 9, 2018, now abandoned.

(30) Foreign Application Priority Data
Feb. 13, 2015 (AU) 2015900484

(51) Int. Cl. G01N 33/574; C07K 16/28; C07K 16/32; G01N 33/68

(52) U.S. Cl. CPC G01N 33/57492; C07K 16/2863; C07K 16/32; G01N 33/6872; G01N 2333/705; G01N 2333/82; G01N 2800/52

(57) ABSTRACT

Disclosed are methods for classifying tumors according to their responsiveness to a therapeutic agent based on the clustering status of a cell surface receptor element to which the therapeutic agent is capable of binding. Also disclosed are 5 methods for stratifying subjects with cancer into treatment subgroups based on this classification as well as methods for treating subjects so stratified.

This application claims priority to Australian Provisional Application No. 2015900484 entitled “Methods for classifying tumors and uses therefor”, filed on 13 Feb. 2015, the entire content of which is hereby incorporated by reference herein.

### FIELD OF THE INVENTION

This invention relates generally to methods for classifying tumors according to their responsiveness to a therapeutic agent based on the clustering status of a cell surface receptor element that is capable of interacting with the therapeutic agent when it is clustered. The present invention also relates to methods for stratifying subjects with cancer into treatment subgroups based on this classification and to methods for treating subjects so stratified.

Bibliographic details of various citations referred to by author in the present specification are listed at the end of the description.

### BACKGROUND OF THE INVENTION

The Human Epidermal growth factor Receptor (HER) family is a group of four receptor tyrosine kinases commonly overexpressed in many cancers of the breast (Abd El-Rehim et al., 2004; Suo et al., 2002; Witton et al., 2003), gastro intestinal tract (Hayashi et al., 1994; Ooi et al., 2004; Porebska et al., 2000), lung (Hirsch et al., 2003), and prostate (Di Lorenzo et al., 2002). For example, 20% to 30% of breast cancers present with amplification of the HER2/neu oncogene, which subsequently overexpress the second member of the HER family, HER2. HER2 positive tumors often demonstrate high metastatic potential, although the development and clinical implementation of targeted anti-cancer therapies such as the monoclonal antibody (mAb) Trastuzumab has demonstrated a benefit in the treatment of HER2 positive cancers (Piccart-Gebhart et al., 2005). Unfortunately, this benefit is limited to less than 35% of patients with HER2 positive breast cancer (Narayan et al., 2009; Wolff et al., 2007), 70% of whom progress to develop therapeutic resistance within the first 12 months of commencing therapy, even when Trastuzumab is used as an adjuvant to chemotherapy (Gajria & Chandarlapaty, 2011; Vu & Claret, 2012). Therefore, there is a clear rationale for understanding the mechanisms which contribute to the development of resistance to anti-HER mAbs in HER positive breast cancers.

HER family signaling activation is usually triggered in response to ligand binding (Olayioye, 2001). While many ligands have been identified for HER1 (EGFR, ErbB1), HER3 (ErbB3) and HER4 (ErbB4), no ligand has been identified for HER2 (Eigenbrot et al., 2010). Instead HER2 appears to be a co-receptor tyrosine kinase that exists in a constitutively active conformation, allowing it to be the preferred dimerization partner of the HER family (Eigenbrot et al., 2010; Graus-Porta, Beerli, Daly, & Hynes, 1997; Wieduwilt & Moasser, 2008). Following homo- or hetero-dimerization, trans-autophosphorylation of the intracellular domains of the receptors occurs. This produces specific docking and activation sites for Important signaling molecule intermediates including those containing phosphotyrosine binding (PTB) and Src homology 2 (SH2) domains. Such intermediates participate in the mitogen-activated protein kinase (MAPK) and phosphatidylinositol 3-kinase (PI3/AKT) pathways (Pinkas-Kramarski et al., 1998; Yarden, 2001). Under normal conditions, such signaling cascades have been reported to play important roles in the regulation of cell survival, proliferation, differentiation and migration, however, receptor over-expression has been demonstrated to promote tumorigenicity (Graus-Porta et al., 1997; Tzahar et al., 1996). As such, regulation and down-modulation of HER signaling is essential to maintain the physiology of the cells in which they are expressed. Additionally, Mellman and Yarden (2013) note that derailed internalization of receptor tyrosine kinases (RTKs) can make major contributions to several hallmarks of cancer, including sustained proliferation of cancer cells, enhanced invasiveness and avoidance of apoptosis.

Therapeutic mAbs are emerging as a prominent category of anti-cancer therapeutic agents because of their ability to sterically hinder the association of the target antigens with other molecules, or by affecting the conformation of the target in a way that may alter its activity (Scott et al., 2012). The over-representation of HER family members as drivers of tumorigenesis led to the development of therapeutic mAbs, such as Trastuzumab (Herceptin®) and Cetuximab (Erbitux®) which bind with high affinity to the extracellular domains of HER2 and HER1 respectively (Blick & Scott, 2007; Nahta et al., 2004). Both Trastuzumab and Cetuximab have been shown to reduce receptor mediated down-stream signaling which has been demonstrated to induce cell cycle arrest and apoptosis in vitro and to facilitate inhibition of tumor growth and angiogenesis in vivo (Izumi et al., 2002; Klos et al., 2003; Komarova et al., 2011; Pueyo et al., 2010; Vincenzi et al., 2006). Like other therapeutic mAbs that bind to cell surface receptors on tumor cells such as pembrolizumab, which is specific for the programmed cell death protein, PD-1, and TRX518, which is specific for glucocorticoid-induced TNFR-related protein (GITR), Trastuzumab and Cetuximab also appear to mediate the induction of antibody dependent cellular cytotoxicity (ADCC) as well as complement dependent cytotoxicity (CDC) against tumor cells in vitro (Mellstedt, 2003, Barok et al., 2007; Patel et al., 2010, Noguchi et al., 2013).

While the fraction antibody binding (Fab) domains of Trastuzumab and Cetuximab are individually distinct, their fraction crystallizable (Fc) domains are identical. The isoform of the Fc domain is important as it can direct specific features of its function in vitro and in vivo (Patel et al., 2010). For example, immune cells such as Natural Killer (NK) cells express FcγRIIIa receptors (Srivastava et al., 2013). These receptors bind IgG1 which triggers immune cell activation and represents the first step in the induction of perforin and granzyme mediated anti-tumor immune responses (Mace et al., 2014). A growing body of evidence suggests that the main therapeutic benefit of mAbs such as Trastuzumab and Cetuximab is derived from their ability to induce targeted immune responses (Barok et al., 2007). The most likely cellular candidate for this response are CD56^{dim} CD16⁺ NK cells which express high levels of FcγRIIIa receptor (Zimmer et al., 2007). In vivo analysis of FcγR-deficient mice treated with Trastuzumab or Cetuximab demonstrated reduced anti-tumor response in comparison to wild type mice (Clynes et al., 2000). Also, ex vivo analysis of tumors removed from patients treated with Trastuzumab have revealed a significant elevation in the level of NK cell infiltration in comparison to tumors analyzed from patients not treated with Trastuzumab (Esendagli et al., 2008). These findings indicate that the therapeutic efficacy of Trastuzumab and Cetuximab involved the induction of cytotoxic immune responses, however, the efficacy of such responses are only maintained as long as tumor cells remain sensitive to the therapies themselves (Ahmad et al., 2014).

Several investigators have reported that cellular distribution and conformation of mAb target receptors are factors that contribute to antibody efficacy in eliciting anti-tumor effects. For example, Scaltriti et al. (2009) suggested that the synergistic anti-tumor efficacy of Trastuzumab combined with the HER1/HER2 tyrosine kinase inhibitor (TKI) Lapatinib (Tykerb®; GlaxoSmithKline) may have been due to the ability of this TKI to stabilize HER2 at the plasma membrane by inhibiting its activity, and in turn enhancing accumulation and presentation of Trastuzumab to immune effector cells which are able to induce antibody mediated anti-tumor activity. Moreover, the present inventors have shown a significant correlation between positive response to anti-HER1 therapeutic mAbs and HER1 trafficking defects that inhibit HER1 internalization ex vivo in head and neck squamous cell carcinoma (HNSCC) tumors (see, International Publication WO 2014/063205). Significantly, the present inventors have also found that inhibition of HER1 endocytosis in vitro with small molecule inhibitors of dynamin restores sensitivity of tumor cells to Cetuximab treatment (see, International Publication WO 2014/063206). Together these models suggest that therapeutic mAb targeted antigen trafficking dynamics significantly contribute to the efficacy of anti-cancer mAb therapies.

### SUMMARY OF THE INVENTION

The present invention arises in part from the unexpected discovery that clustering of receptor elements on the surface of tumor cells is a better surrogate marker of tumor cell responsiveness or non-responsiveness to receptor antagonist therapy, than currently known methods. In particular, the present inventors have found that despite significant HER expression on the surface of some HER positive tumors, these tumors nevertheless have reduced or impaired sensitivity to HER antagonist therapy (e.g., using a HER ligand such as an anti-HER antibody). Surprisingly, further analysis of HER expression on the surface of these tumors revealed that HER clustering is significantly reduced or impaired, as compared to HER antagonist sensitive tumors. By contrast, HER positive tumors with unimpaired receptor clustering were found to be responsive to HER antagonist therapy. Based on these findings, the present inventors propose that the clustering status of receptors at the surface of tumor cells generally correlates with tumor cell responsiveness or non-responsiveness to cognate receptor ligand (e.g., antagonist or agonist) therapies. As such, methods and kits as well as associated reagents and compositions are proposed for classifying tumors into different clinical subtypes or for stratifying tumor-affected subjects into different treatment subgroups according to the receptor clustering status of the tumors. These methods, kits, compositions and reagents enable better selection of treatment of tumors and affected subjects, as described hereafter.

Thus, the present invention addresses the problem of distinguishing between receptor antagonist responders and non-responders by determining the degree of receptor clustering in tumors from cancer-affected subjects. This represents a significant advance over current technologies for the management of cancers including HER positive cancers, and permits improved selection of patients for treatment with receptor ligand (e.g., antagonist or agonist) therapies in order to predict an increased likelihood of response to those therapies.

Accordingly, in one aspect, the present invention provides methods for classifying a tumor's responsiveness to a therapeutic agent that is a ligand of a cell surface receptor, wherein the tumor is positive for cell surface receptor elements. These methods generally comprise, consist or consist essentially of determining the clustering status of cell surface receptor elements in a sample of the tumor, wherein the clustering status is used to classify the tumor's responsiveness to the therapeutic agent. In some embodiments, the methods further comprise determining the distribution of the cell surface receptor elements on a tumor cell of the sample, which tumor cell is positive for the cell surface receptor elements, and correlating the determined distribution of the cell surface receptor elements on the tumor cell with the formation of receptor clusters, thereby determining the clustering status of the cell surface receptor elements. Generally, the methods comprise determining the presence of receptor clusters on a tumor cell of the sample, which tumor cell is positive for the cell surface receptor elements, wherein individual clusters comprise at least two of the cell surface receptor elements. The present invention contemplates any cell surface receptor in the practice of the present invention, which is capable of binding a therapeutic agent (e.g., a therapeutic ligand such as a therapeutic antibody). Representative cell surface receptors are described below and these include for example HER family members such as HER1 and HER2, programmed cell death proteins such as PD-1 and tumor necrosis factor receptor superfamily members such as GITR.

Another aspect of the present invention provides methods for stratifying a subject with a cell surface receptor element-positive cancer into a treatment subgroup selected from responder and non-responder to a therapeutic agent that binds to the cell surface receptor. These methods generally comprise, consist or consist essentially of classifying a cell surface receptor element-positive tumor of the subject according to the tumor classification methods broadly described above and elsewhere herein, and identifying the subject as a responder or non-responder to the therapeutic agent according to the clustering status of the cell surface receptor element in a sample of the tumor. In some embodiments, these methods further comprise taking a tumor sample from the subject for use in the stratification.

In some embodiments, all or a part of the tumor classification or patient stratification methods broadly described above and elsewhere herein are performed by a processing system.

In another aspect, the present invention provides methods for treating a subject with a cell surface receptor element-positive cancer. These methods generally comprise, consist or consist essentially of exposing the subject to a therapy based on the results of a stratification determining method as broadly described above and elsewhere herein, which stratifies the subject into a treatment subgroup selected from responder and non-responder to a therapeutic agent that binds to a cell surface receptor that comprises cell surface receptor elements, and administering the therapeutic agent to the subject on the basis that the subject is stratified into the responder subgroup or administering a cancer therapy other than the therapeutic agent to the subject on the basis that the subject is stratified into the non-responder subgroup. In some embodiments, the methods further comprise taking a tumor sample from the subject and stratifying the subject according to the stratification determining method. In other embodiments, the methods further comprise sending a tumor sample from the subject to a laboratory at which the stratification is determined according to the stratification determining method. In preferred embodiments, the therapeutic agent is an antibody.

In specific embodiments, the cell surface receptor is an epidermal growth factor receptor family member (e.g., EGFR (HER1), ErbB2 (HER2), ErbB3, ErbB4) and the therapeutic agent is selected from antibodies that bind to the EGFR family member. In other embodiments, the cell surface receptor is a programmed cell death protein family member (e.g., PD-1, PD-2, PD-3, PD-4, PD-5, PD-6) and the therapeutic agent is selected from antibodies that bind to the programmed cell death protein family member. In still other embodiments, the cell surface receptor is a TNFR superfamily member such as GITR and the therapeutic agent is selected from antibodies that bind to the TNFR member.

In some embodiments, the methods further comprise co-administering an ancillary cancer therapy to the subject, illustrative examples of which include radiotherapy, surgery, chemotherapy, hormone ablation therapy, pro-apoptosis therapy and immunotherapy other than the antibody.

Still another aspect of the present invention provides kits for classifying a tumor's responsiveness to a therapeutic agent, wherein the tumor is a cell surface receptor element-positive tumor. These kits comprise a reagent for use in the tumor classification or patient stratification methods as broadly described above and elsewhere herein. In specific embodiments, the reagent is a ligand of the cell surface receptor, which is suitably labeled. In illustrative examples, the ligand is a labeled or non-labeled antibody that binds to the cell surface receptor.

### BRIEF DESCRIPTION OF THE DRAWINGS

FIG. 1 is a graphical representation showing HER2 total expression by ELISA of breast cancer cell lines SKB3, BT474, HCC1569 and MDA-MB-231. Notably, some cells lines including SKBR3 show high levels of HER2 total expression while MDA-MB-231 is HER2 negative.

FIG. 2 is a graphical representation showing multi-variant FACS analysis of ADCC activity of Herceptin for various cell lines in the presence of fresh PBMC. (A) SKB3, (B) BT474 (C) and (D) HCC1569. Statistical analyses conducted using Students Two-Tailed T-tests. ***P<0.0001, ***P<0.001, **P<0.01, ns P>0.05. Data presented as SEM, n=3. Total specific target cell deaths ranging below (- - -) would model in vivo insensitivity, while those above (- - -) would likely model in vivo sensitivity. Although BT474 and HCC1569 cell lines express similar amounts of HER2, their respective killing levels are very dissimilar.

FIG. 3 is a graphical representation showing that HER2 cell surface level correlates better with sensitivity to Trastuzumab mediated immune-dependent tumor cell death, than total expression levels of the receptor. (A) Comparison of the relative MFI of SKBR3, BT474, HCC1569, MCF7, MDA-MB-468, and MDA-MB-231 treated with 60 μg/mL of Trastuzumab for 4 hours and labeled with secondary anti-human Alexa⁶⁴⁷, with % specific cell death of the same cell lines treated with 60 μg/ml of Trastuzumab. R²=0.96, P<0.001. Data are presented as SEM. MFI n=1, Specific cell death n=3. (B) Comparison of relative total HER2 expression level of SKBR3, BT474, HCC1569, MCF7, MDA-MB-468, and MDA-MB-231 cells with % specific cell death of the same cell lines treated with 60 μg/mL of Trastuzumab. R₂=0.69, P<0.05. Data are presented as SEM, n=3.

FIG. 4 is a graphical representation showing HER2 surface expression of breast cancer cell lines SKB3, BT474, HCC1569 and HER2-negative MDA-MB-231 as well as the ADCC activity of Herceptin for each cell line in the presence and absence of a dynamin-mediated endocytosis inhibitor (Dyngo4a). (A) FACS analysis shows that Dyngo4a does not increase surface levels of HER2 on the cell lines. However, Dyngo4a increased the ADCC activity of Herceptin for the HER2-expressing cell lines SKB3 (B), BT474 (C) and HCC1569 (D). Of note, the dynamin inhibitor significantly increased responsiveness of Herceptin-resistant cell line HCC1569 to Herceptin mediated ADCC.

FIG. 5 is a graphical representation showing HER1 surface expression of breast cancer cell lines MDA-MB-468, MDA-MB-231 and SKB3 as well as the ADCC activity of Cetuximab for each cell line in the presence and absence of a Dyngo4a. (A) FACS analysis shows that Dyngo4a does not increase surface levels of HER1 on the cell lines. However, Dyngo4a increased the ADCC activity of Cetuximab for the HER1-expressing cell lines MDA-MB-468 (B), MDA-MB-231 (C) and SKB3 (D). Significantly, Dungo4a markedly increased responsiveness of Cetuximab-resistant cell triple negative cell line MDA-MB-231 to Cetuximab mediated ADCC.

FIG. 6 is a graphical representation showing that IgG1 mediated ADCC is via CD56 positive NK cells.

FIG. 7 is a photographic representation showing that clathrin inhibitor, Pitstop®-2, but not dynamin inhibitor, chlorpromazine, inhibits CCV formation. (A-C): Confocal imaging. A431 cells were pretreated at 37° C. for 25 min. (control, A) with Pitstop®-2 (B) or chlorpromazine (C), before addition of EGF-Alexa¹⁸⁸ (green) for a further five minutes. The cells were fixed and labeled with anti-clathrin mouse monoclonal primary antibody with anti-mouse Alexa⁵⁹⁴ secondary antibody (red). The nuclei were labeled with DAPI (blue). The images were acquired using a Zeiss 510 Meta confocal with a 63 Å˜ objective (scale bars, 20 μm). (D-I): 3D-SIM super-resolution imaging, Chlorpromazine causes accumulation of clathrin and EGFAlexa⁴⁸⁸ at the cell surface. The upper panels (D-F) show a single cell 3-D reconstruction and the lower panels (G-I) show a 3D volume projection of a small area of the cell periphery (note that panel f is a larger region of the cell), In control cells (D, G) EGF-Alexa⁴⁸⁸ (green) was found throughout the cell in endosomal structures and at cell periphery. Similarly clathrin (anti-mouse Alexa-594 to anti-clathrin: Red) was localized to the cell periphery and endosomal structures, some of which were in close proximity to EGF-Alexa⁴⁸⁸ ligand (D, G arrow). After Pitstop®-2 treatment (E, H) EGFAlexa⁴⁸⁸ no longer accumulated within endosomal structures, and only a low level of both EGFAlexa⁴⁸⁸ and clathrin can be seen at the plasma membrane with little association. After chlorpromazine (F, I) EGF-Alexa⁴⁸⁸ localized to the plasma membrane and was much brighter than with Pitstop®-2, with little endosomal EGF-Alexa⁴⁸⁸. Clathrin staining accumulated in the cell periphery, some co-localizing with EGF-Alexa⁴⁸⁸ ligand (I; arrows). The lower panels (3D volume projections of a small area of the cell periphery; (G-I) show the difference in EGF-Alexa⁴⁸⁸ and clathrin localization following treatments; control cells (D) have EGF-488 and clathrin accumulated throughout the cell sometimes in close proximity (arrow), Pitstop®-2 treated cells have less EGFAlexa⁴⁸⁸ and clathrin at the cell surface and minimal association whereas chlorpromazine-treated cells have EGF-Alexa⁴⁸⁸ accumulated at the cell surface with clathrin in close proximity, forming almost a layer under the EGF-Alexa488 ligand.

Some figures and text contain color representations or entities. Color illustrations are available from the Applicant upon request or from an appropriate Patent Office. A fee may be imposed if obtained from a Patent Office.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, illustrative methods and materials are described. For the purposes of the present invention, the following terms are defined below.

The use of numerical values in the various ranges specified in this application, unless expressly indicated otherwise, are stated as approximations as though the minimum and maximum values within the stated ranges were both preceded by the word “about.” In this manner, slight variations above and below the stated ranges (e.g., less than or equal to 10%, 9%, 8%, 7%, 6%, 5%, 4%, 3%, 2%, 1%) can be used to achieve substantially the same results as values within the ranges. Also, the disclosure of these ranges is intended as a continuous range including every value between the minimum and maximum values.

The articles “a” and “an” are used herein to refer to one or to more than one (i.e. to at least one) of the grammatical object of the article. By way of example, “an element” means one element or more than one element.

The term “antibody” herein is used in the broadest sense and specifically covers monoclonal antibodies, polyclonal antibodies, multispecific antibodies (e.g., bispecific antibodies), antibody fragments, or any other antigen-binding molecule so long as they exhibit the desired biological activity.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target (e.g., a target antigen), wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al. (1991) Nature 352:624-628; Marks et al. (1991) J. Mol. Biol. 222:581-597; Sidhu et al. (2004) J. Mol. Biol. 338(2):299-310; Lee et al. (2004) J. Mol. Biol. 340(5): 1073-1093; Fellouse (2004) Proc. Nat. Acad. Sci. USA 101(34):12467-12472; and Lee et al. (2004) J. Immunol. Methods 284(1-2): 119-132, and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al. (1993) Proc. Natl. Acad. Sci. USA 90:2551; Jakobovits et al. (1993) Nature 362:255-258; Bruggemann et al. (1993) Year in Immuno. 7:33; U.S. Pat. Nos. 5,545,806; 5,569,825; 5,591,669 (all of GenPharm); U.S. Pat. No. 5,545,807; WO 1997/17852; U.S. Pat. Nos. 5,545,807; 5,545,806; 5,569,825; 5,625,126; 5,633,425; and U.S. Pat. No. 5,661,016; Marks et al. (1992) Bio/Technology 10: 779-783; Lonberg et al. (1994) Nature 368: 856-859; Morrison (1994) Nature, 368: 812-813; Fishwild et al. (1996) Nature Biotechnology 14: 845-851; Neuberger (1996) Nature Biotechnology 14: 826; and Lonberg and Huszar (1995) Intern. Rev. Immunol. 13: 65-93).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA 81:6851-6855). Chimeric antibodies of interest herein include “primatized” antibodies comprising variable domain antigen-binding sequences derived from a non-human primate (e.g., Old World Monkey, Ape etc.) and human constant region sequences, as well as “humanized” antibodies.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. Biol. 2:593-596.

“Antibody fragments” comprise a portion of an intact antibody, suitably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

An antibody “that binds” an antigen of interest (e.g., a tumor surface antigen such as HER1 or HER2) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody, oligopeptide or other organic molecule to its particular target protein as determined by fluorescence activated cell sorting (FACS) analysis or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. In some embodiments, the primary cells for mediating ADCC, NK cells, express FcγRIII only, whereas monocytes express FcγRI, FcγRII and FcγRIII, FcR expression on hematopoietic cells in summarized is Table 3 on page 464 of Ravetch and Kinet, (1991) Annu. Rev. Immunol. 9:457-92. To assess ADCC activity of a molecule of interest, an in vitro ADCC assay, such as that described in U.S. Pat. No. 5,500,362 or 5,821, 337 may be performed. Useful effector cells for such assays include peripheral blood mononuclear cells (PBMC) and Natural Killer (NK) cells. Alternatively, or in addition, ADCC activity of the molecule of interest may be assessed in vivo, e.g., in a animal model such as that disclosed in Clynes et al. (1998) Proc. Natl. Acad. Sci. USA 95:652-656.

The term “complement-mediated cytotoxicity” refers to cytotoxicity that requires presence and/or activity of at least one component of the complement system. In some embodiments, complement-mediated cytotoxicity requires one or more components of the classical pathway of the complement system; in some embodiments, complement-mediated cytotoxicity requires one or more components of the alternative pathway.

By “clustering”, and grammatical equivalents used herein, is meant any reversible or irreversible association of one or more cell surface receptor elements. Clusters can be made up of 2, 3, 4, etc., receptor elements. Clusters of two receptor elements are termed dimers. Clusters of 3 or more receptor elements are generally termed oligomers, with individual numbers of clusters having their own designation, for example, a cluster of 3 receptor elements is a trimer, a cluster of 4 receptor elements is a tetramer, etc.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “effective amount”, in the context of treating or preventing a disease or condition (e.g., a cancer) is meant the administration of an amount of active agent to a subject, either in a single dose or as part of a series or slow release system, which is effective for the treatment or prevention of that disease or condition. The effective amount will vary depending upon the health and physical condition of the subject and the taxonomic group of individual to be treated, the formulation of the composition, the assessment of the medical situation, and other relevant factors.

The term “cell surface receptor element”, as used herein, refers to a receptor element that is displayed on the surface of a cell. In most cases, the receptor element will be located in or on the plasma membrane of the cell such that at least part of this receptor element remains accessible from outside the cell in tertiary form. A non-limiting example of a cell surface receptor element that is located in the plasma membrane is a transmembrane protein comprising, in its tertiary conformation, regions of hydrophilicity and hydrophobicity. Here, at least one hydrophobic region allows the cell surface receptor element to be embedded, or inserted in the hydrophobic plasma membrane of the cell while the hydrophilic regions extend on either side of the plasma membrane into the cytoplasm and extracellular space, respectively. A cell surface receptor element is capable of interacting with a ligand, e.g., hormone, peptide or small molecule, suitably when clustered, resulting in the propagation of an intra- or extracellular signal. The term “cell surface receptor element” includes within its scope individual receptor polypeptides or monomers as well as receptor polypeptides in multimeric form (e.g., dimers, trimers, etc.).

As used herein a “cell surface receptor ligand sensitive tumor” refers to a cell surface receptor element positive tumor in which at least 30%, 40%, 50%, 60%, 70%, 80%, 90%, 95% or up to 100% of cell surface receptor element-expressing cells in the tumor have unimpaired clustering of the cell surface receptor elements. Thus, a “HER antagonist sensitive tumor” refers to a HER positive tumor in which at least 30%, 40%, 50%, 60%, 70%, 80%, 90%, 95% or up to 100% of HER-expressing cells in the tumor have unimpaired clustering of HER.

As used herein a “cell surface receptor ligand resistant tumor” refers to a cell surface receptor element positive tumor in which at least 30%, 40%, 50%, 60%, 70%, 80%, 90%, 95% or up to 100% of cell surface receptor element-expressing cells in the tumor have impaired clustering of the cell surface receptor elements. Thus, a “HER antagonist resistant tumor” refers to a HER negative tumor or a HER positive tumor in which at least 30%, 40%, 50%, 60%, 70%, 80%, 90%, 95% or up to 100% of cells in the tumor have an impaired clustering of HER.

The term “cell surface receptor element-expressing cell” as used herein refers to cells that express a cell surface receptor polypeptide. “Cell surface receptor expression” refers to conversion of the information encoded in by the gene encoding the cell surface receptor into messenger RNA (mRNA) and then to the cell surface receptor polypeptide.

As used herein, a tumor cell or cell population is “positive for” a specific cell surface receptor element or “positive” when the specific cell surface receptor element is detected on the tumor cell or cell population. Similarly, a tumor cell or cell population is “negative for” a specific cell surface receptor element, or “negative” when the specific cell surface receptor element is not detected on the tumor cell or cell population. Whether a cell or cell population is positive or negative for a particular cell surface receptor can be determined by standard methods known in the art. For example, a tumor cell negative for a cell surface receptor element refers to the absence of significant staining of the tumor cell with a specific antibody (e.g., less than 10%, 5%, 1%, or less) above an isotype matched control antibody (i.e., background), whereas a tumor cell positive for the cell surface receptor element refers to staining of the tumor cell (e.g., at least 10%, 15%, 20%, 25%, 30%, or more) above the isotype control. Likewise a cell population negative for a cell surface receptor element refers to the absence of significant staining of the cell population with the specific antibody (e.g., less than 10%, 5%, 1%, or less) above the isotype control, and positive refers to uniform staining of the cell population (e.g., at least 10%, 15%, 20%, 25%, 30%, or more) above the isotype control. In some embodiments, a cell population positive for a cell surface receptor element refers to a percentage of cells that exhibit the cell surface receptor element above background; e.g., at least 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any integer % between 50 and 100% of cells that exhibit the cell surface receptor element above background, when compared to a reference cell population.

The term “cell surface receptor element-positive tumor” as used herein refers to a tumor that contains at least 1%, particularly at least 2%, 3%, 4% or 5%, particularly at least 10%, 20%, 30%, 40%, 50%, 60%, 70%, 80%, 90% cell surface receptor element-expressing cells, detected by a standard assay known in the art; e.g. by an immunoassay such as an immunohistochemistry test. In specific embodiments, the cell surface receptor element-positive cells overexpress the cell surface receptor element. By “overexpression of a cell surface receptor element” and the like is intended to mean an abnormal level of expression of a cell surface receptor element in a cell from a tumor within a specific tissue or organ of a patient relative to the level of expression in a normal cell from that tissue or organ. Patients having a cancer characterized by overexpression of a cell surface receptor element can be determined by standard assays known in the art, as for example noted above. Cancers characterized by cell surface receptor element-positive tumor are referred to herein as “cell surface receptor element-positive cancers.” In specific embodiments, the cell surface receptor element is a HER (e.g., HER1, Her2, etc.). Thus, the term “HER-positive tumor” as used herein refers to a tumor that contains at least 1%, particularly at least 2%, 3%, 4% or 5%, particularly at least 10%, 20%, 30%, 40%, 50%, 60%, 70%, 80%, 90% HER-expressing cells, detected e.g. by an immunohistochemistry test such as, for example, the EGFR pharmDx kit (DAKO North America, Inc., Carpinteria, CA, USA), and the HercepTest® (DAKO North America, Inc., Carpinteria, CA, USA). In specific embodiments, the HER positive cells overexpress a specific HER (e.g., HER1, Her2, etc.). By “overexpression of HER” and the like is intended to mean an abnormal level of expression of a HER (e.g., HER1, Her2, etc.) in a cell from a tumor within a specific tissue or organ of a patient relative to the level of expression in a normal cell from that tissue or organ. Patients having a cancer characterized by overexpression of the HER can be determined by standard assays known in the art, as for example noted above. Cancers characterized by HER-positive tumor are referred to herein as “HER-positive cancers.”

As used herein, the terms “label” and “detectable label” refer to a molecule capable of being detected, where such molecules include, but are not limited to, radioactive isotopes, fluorescers (fluorophores), chemiluminescers, chromophores, enzymes, enzyme substrates, enzyme cofactors, enzyme inhibitors, dyes, metal ions, metal sols, ligands (e.g., biotin, avidin, streptavidin or haptens), intercalating dyes and the like. The term “fluorescer” or “fluorophore” refers to a substance or a portion thereof which is capable of exhibiting fluorescence in a detectable range.

The term “ligand” as used herein refers to a naturally occurring or synthetic compound that binds to a cell surface receptor (e.g., a HER such as HER1 or HER2). Upon binding to a receptor, ligands generally lead to the modulation of activity of the receptor. The term is intended to encompass naturally occurring compounds, synthetic compounds and/or recombinantly produced compounds. As used herein, this term can encompass agonists, antagonists, and inverse agonists.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomolgous monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice, rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars), marine mammals (e.g., dolphins, whales), reptiles (e.g., snakes, frogs, lizards), and fish. In specific embodiments, the subject is a primate such as a human. However, it will be understood that the aforementioned terms do not imply that symptoms are present.

As used herein, the terms “prevent”, “prevented”, or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it. In preferred embodiments, the disease or condition is a cancer.

As used herein, in the context of a cancer, the term “responder” refers to a patient who exhibits or is more likely to exhibit a beneficial clinical response following treatment with a therapeutic agent that binds to the cell surface receptor. By contrast, the term “non-responder”, as used herein, refers to a patient who is does not exhibit or is less likely to be exhibit a beneficial response following treatment with a therapeutic agent that binds to the cell surface receptor. As used herein in the context of patient response to treatment with a therapeutic agent, the terms “beneficial response”, “beneficial patient response”, and “clinically beneficial response”, “clinical benefit”, and the like, are used interchangeably and refer to favorable patient response to a drug as opposed to unfavorable responses, i.e., adverse events. In individual patients, beneficial response can be expressed in terms of a number of clinical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment. Evaluation of patients in assessing symptoms and/or severity of the disease may be carried out by various methods, which are known in the art. The evaluation may take into account numerous criteria, as determined by suitable biochemical, physiological, and/or behavioral factors.

As used herein, the terms “treatment”, “treating”, and the like, refer to administering an agent, or carrying out a procedure (e.g., radiation, a surgical procedure, etc.) to obtain a desired pharmacologic and/or physiologic effect. The effect may be prophylactic in terms of completely or partially preventing a disease or symptom thereof and/or may be therapeutic in terms of effecting a partial or complete cure for a disease and/or symptoms of the disease. The effect may be therapeutic in terms of a partial or complete cure for a disease or condition (e.g., a cancer) and/or adverse effect attributable to the disease or condition. These terms also cover any treatment of a condition or disease in a mammal, particularly in a human, and include: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; (c) relieving the disease, i.e., causing regression of the disease; (d) reducing the severity of a symptom of the disease and/or (e) reducing the frequency of a symptom of the disease or condition.

The term “tumor”, as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth and include As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, colorectal cancer, breast cancer, ovarian cancer, lung cancer, prostate cancer, hepatocellular cancer, gastric cancer, pancreatic cancer, cervical cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer. In an exemplary embodiment, the cancer is squamous cell carcinoma.

The term “tumor sample” as used herein means a sample comprising tumor material obtained from a cancerous patient. The term encompasses clinical samples, for example tissue obtained by surgical resection and tissue obtained by biopsy, such as for example a core biopsy or a fine needle biopsy. The term also encompasses samples comprising tumor cells obtained from sites other than the primary tumor, e.g., circulating tumor cells, as well as well as preserved tumor samples, such as formalin-fixed, paraffin-embedded tumor samples or frozen tumor samples. The term encompasses cells that are the progeny of the patient's tumor cells, e.g., cell culture samples derived from primary tumor cells or circulating tumor cells. The term encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

Each embodiment described herein is to be applied mutatis mutandis to each and every embodiment unless specifically stated otherwise.

### 2. Abbreviations

| | |
|---|---|
| Ab | Antibody |
| ADCC | Antibody Dependent Cellular Cytotoxicity |
| CDC | Complement Dependent Cytotoxicity |
| EGF | Epidermal Growth Factor |
| EGFR | Epidermal Growth Factor Receptor |
| FACS | Fluorescence-Activated Cell Sorting |
| FcγR | Fcγ Receptor |
| GPCR | G-Protein-Coupled Receptor |
| HNSCC | Head and Neck Squamous Cell Carcinoma |
| IF | Immunofluorescence |
| IHC | Immunohistochemistry |
| IL | Interleukin |
| mAb | Monoclonal Antibody |
| MAPK | Mitogen-Activated Protein Kinase |
| MHC | Major Histocompatibility Complex |
| NK | Natural Killer |
| PBMC | Peripheral Blood Mononuclear Cell |
| RTK | Receptor Tyrosine Kinases |
| SCC | Squamous Cell Carcinoma |

### 3. Methods for Classifying Cell Surface Receptor Element Positive Tumors

The present invention provides methods for classifying tumors into therapeutic agent responsive and therapeutic agent non-responsive subtypes, wherein the therapeutic agent is a ligand of a cell surface receptor, and wherein the tumor is positive for cell surface receptor elements. In accordance with the present invention, these methods involve analyzing the clustering status of the cell surface receptor. A detected unimpaired cell surface receptor element clustering, suitably relative to a control classifies the tumor as responsive to the therapeutic agent, whereas a detected impaired or abrogated cell surface receptor element clustering, suitably relative to a control, classifies the tumor as non-responsive to the therapeutic agent. In some embodiments, the tumor is classified as responsive to the therapeutic agent when it has unimpaired clustering of cell surface receptor elements; suitably when at least 40% (and at least 41% to at least 99% and all integer percentages in between) of the cell surface receptor elements on a cell surface receptor element positive tumor cell are present in clusters. In other embodiments, the tumor is classified as non-responsive to the therapeutic agent when it has impaired clustering of cell surface receptor elements; suitably when less than 40% (and less than 39% to at less than 1% and all integer percentages in between) of the cell surface receptor elements on a cell surface receptor element positive tumor cell are present in clusters.

Suitably, the clustering status is determined relative to a control. In illustrative examples of this type, the tumor is classified as responsive to the therapeutic agent when the level of clustering of the cell surface receptor elements on a cell surface receptor element positive tumor cell of the sample corresponds to the level of clustering of the cell surface receptor elements on a control cell surface receptor element positive tumor cell that is responsive to the therapeutic agent (“a therapeutic agent responsive control tumor cell”). This level of clustering corresponds to an unimpaired cell surface receptor element clustering status. The therapeutic agent responsive control tumor cell can natively display unimpaired clustering of cell surface receptor elements or can have clustering of cell surface receptor elements stimulated or restored by exposing the tumor cell to a dynamin-dependent endocytosis inhibitor.

In other illustrative examples, the tumor is classified as non-responsive to the therapeutic ligand when the level of clustering of the cell surface receptor elements on a cell surface receptor element positive tumor cell of the sample corresponds to the level of clustering of the cell surface receptor elements on a control cell surface receptor element positive tumor cell that is non-responsive to the therapeutic agent (“a therapeutic agent non-responsive control tumor cell”). This level of clustering corresponds to an impaired cell surface receptor element clustering status. The therapeutic agent non-responsive control tumor cell can natively display impaired clustering of the cell surface receptor element or can have clustering of the cell surface elements reduced or abrogated by exposing the tumor cell to a clathrin-dependent endocytosis inhibitor.

In certain embodiments, the methods comprise contacting the tumor sample with a ligand of the cell surface receptor, which is suitably labeled. The ligand can be the therapeutic agent or another ligand of the cell surface receptor. In some embodiments, the ligand comprises an Fc region of an immunoglobulin, which suitably binds with an immune effector cell (e.g., an immune effector cell that mediates at least in part ADCC or CDC). In representative examples of this type, the methods may further comprise measuring an ADCC activity or CDC activity of the ligand. For instance, a tumor may be classified as responsive to the therapeutic ligand when the level of clustering of the cell surface receptor elements on a tumor cell of the sample corresponds to the level of clustering of the cell surface receptor elements on a therapeutic agent responsive control tumor cell and when the ADCC or CDC activity of the ligand on the sample cell surface receptor element positive tumor cell is at least 30% (and at least 31% to at least 99% and all integer percentages in between) of the ADCC or CDC activity of the ligand on the therapeutic agent responsive control tumor cell. In specific embodiments, the ADCC or CDC activity of the ligand on the sample tumor cell is at least 31%, 32%, 33%, 34%, 35%, 36%, 37%, 38%, 39%, 40% of the ADCC or CDC activity of the ligand on the therapeutic agent responsive control tumor cell. Alternatively, a tumor may be classified as non-responsive to the therapeutic ligand when the level of clustering of the cell surface receptor elements on a sample cell surface receptor element positive tumor cell corresponds to the level of clustering of the cell surface receptor elements on a therapeutic agent non-responsive control tumor cell and when the ADCC or CDC activity of the ligand on the sample tumor cell is less than 30% (and less than 29% to less than 1% and all integer percentages in between) of the ADCC or CDC activity of the ligand on the therapeutic agent responsive control tumor cell.

Thus, the present invention is predicated on determining the presence, absence or level of clustered cell surface receptor elements on a tumor cell to thereby determine the responsiveness of the tumor cell to a therapeutic agent that binds with the cell surface receptor. The clusters can be made up of identical receptor elements or different receptor elements. Clusters of identical receptor elements are termed “homo” clusters, while clusters of different receptor elements are termed “hetero” clusters. Accordingly, a cluster can be a homomultimer or heteromutlimer. For example, HER1, HER2, HER3 and HER4 can each form homodimers or heterodimers with other HER family members. In other embodiments, the cluster is a homotrimer, as in the case, for example, of TNFα, or a heterotrimer such the one formed by membrane-bound and soluble CD95 to modulate apoptosis. In further embodiments the cluster is a homo-oligomer, as in the case of thyrotropin releasing hormone receptor, or a hetero-oligomer, as in the case of TGFβ1.

Clustering can occur in a variety of ways, illustrative examples of which include cell surface receptors that are stimulated to cluster by binding to ligands, or by binding to other surface molecules.

In specific embodiments, cell surface receptor elements cluster upon ligand binding. As is known in the art, these receptor elements can have a variety of forms, but in general they comprise at least three domains. First, these receptors have a ligand binding domain, which can be oriented either extracellularly or intracellularly. Next, these receptors have a membrane-binding domain, which can take the form of a transmembrane domain or a lipid modification, such as myristylation, to one of the receptor's amino acids which allows for membrane association when the lipid inserts itself into the lipid bilayer. Finally, the receptor has an signaling domain, which is responsible for propagating the downstream effects of the receptor.

The cell surface receptor element-positive tumor can be selected from pre-cancerous, cancerous, non-metastatic or metastatic tumors. In various embodiments, the cell surface receptor element-positive tumor is associated with a cancer selected from carcinoma, lymphoma, blastoma, sarcoma, neuroendocrine tumors, mesothelioma, schwannoma, meningioma, adenocarcinoma, melanoma, leukemia, and lymphoid malignancies. In specific embodiments, the cancer is selected from lung cancer, hepatocellular cancer, gastric or stomach cancer, pancreatic cancer, glioblastoma, cervical cancer, ovarian cancer, liver cancer, bladder cancer, hepatoma, breast cancer, colon cancer, rectal cancer, colorectal cancer, endometrial and uterine carcinoma, salivary gland carcinoma, kidney cancer, prostate cancer, vulval cancer, thyroid cancer, hepatic carcinoma, anal carcinoma, penile carcinoma, testicular cancer, esophageal cancer, tumors of the biliary tract, and head and neck cancer. In specific embodiment, the tumor is of an epithelial origin, non-limiting examples of which include cancer of the lung, colon, prostate, ovary, breast, and skin. Suitably, the cell surface receptor-positive tumor is a tumor of an epithelial origin, illustrative examples of which include cancers of lung, colon, prostate, ovary, breast, or skin (e.g., squamous cell carcinoma (SCC).

### 3.1 Cell Surface Receptor Elements

Cell surface receptors elements are suitably selected from growth factor receptors, cytokine receptors, hormone receptors, tumor differentiation antigens and cluster of differentiation molecules.

Non-limiting examples of growth factor receptors include: epidermal growth factor receptor family members (e.g., EGFR (HER1), ErbB2 (HER2), ErbB3, ErbB4), insulin receptor (IR), insulin-like growth factor receptor (IGFR), vascular endothelial growth factor receptor (VEGFR), tumor necrosis factor receptor (TNFR) superfamily member (e.g., TNFRSF1A, TNFRSF1B, GITR), fibroblast growth factor receptor (FGFR), hepatocyte growth factor receptor (HGFR), platelet derived growth factor receptor (PDGFR), and ephrin receptor (EPH) family members (e.g., EPH-A1, EPH-A2, etc.).

Illustrative examples of cytokine receptors include: cytokine receptor common gamma chain, interleukin-10 receptor alpha chain, interleukin-10 receptor beta chain, interleukin-12 receptor beta-1 chain, interleukin-12 receptor beta-2 chain, interleukin-13 receptor alpha-1 chain, interleukin-13 receptor alpha-2 chain, interleukin-17 receptor, interleukin-17b receptor, interleukin 21 receptor precursor, interleukin-1 receptor, type I, interleukin-1 receptor, type II, interleukin-2 receptor alpha chain, interleukin-2 receptor beta chain, interleukin-3 receptor alpha chain, interleukin-4 receptor alpha chain, interleukin-5 receptor alpha chain, interleukin-6 receptor alpha chain, interleukin-6 receptor beta chain, interleukin-7 receptor alpha chain, high affinity interleukin-8 receptor a, high affinity interleukin-8 receptor b, interleukin-9 receptor, interleukin-18 receptor 1, TNF-related apoptosis-inducing ligand, toll-like receptor 1, toll-like receptor, toll-like receptor 5, cx3c chemokine receptor 1, C-X-C chemokine receptor type 3, C-X-C chemokine receptor type 4, C-X-C chemokine receptor type 5, C-X-C chemokine receptor type 6, chemokine binding protein 2, C-C chemokine receptor type 1, C-C chemokine receptor type 2, C-C chemokine receptor type 3, C-C chemokine receptor type 4, C-C chemokine receptor type 5, C-C chemokine receptor type 6, C-C chemokine receptor type 8, C-C chemokine receptor type, C-C chemokine receptor type 10, C-C chemokine receptor type 11, chemokine receptor-like 1, chemokine receptor-like 2, and chemokine XC receptor 1. In specific embodiments, the cell surface receptor is an epidermal growth factor receptor family member (e.g., EGFR (HER1), ErbB2 (HER2), ErbB3, ErbB4).

Non-limiting examples of cluster of differentiation (also known as cluster of designation) (CD) molecules include: CD1a, CD1b, CD1c, CD1d, CD1e, CD2, CD3d, CD3e, CD3g, CD4, CD5, CD6, CD7, CD8a, CD8b, CD9, CD10, CD11a, CD11b, CD11c, CD11d, CDw12, CD14, CD16a, CD16b, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD44, CD45, CD46, CD47, CD48, CD49a, CD49b, CD49c, CD49d, CD49e, CD49f, CD50, CD51, CD52, CD53, CD54, CD55, CD56, CD57, CD58, CD59, CD61, CD62E, CD62L, CD62P, CD63, CD64, CD66a, CD66b, CD66c, CD66d, CD66e, CD66f, CD68, CD69, CD70, CD71, CD72, CD74, CD79a, CD79b, CD80, CD81, CD82, CD83, CD84, CD85a, CD85c, CD85d, CD85e, CD85f, CD85g, CD85h, CD85i, CD85j, CD85k, CD86, CD87, CD88, CD89, CD90, CD91, CD92, CD93, CD94, CD95, CD96, CD97, CD98, CD99, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD144, CD146, CD147, CD148, CD150, CD151, CD152, CD153, CD154, CD 155, CD156a, CD156b, CD157, CD158a, CD158b1, CD158b2, CD158c, CD158d, CD158e, CD158f1, CD158g, CD158h, CD158i, CD158j, CD158k, CD158z, CD159a, CD159c, CD160, CD161, CD162, CD163, CD163b, CD164, CD165, CD166, CD167a, CD167b, CD168, CD169, CD170, CD171, CD172a, CD172b, CD172g, CD173, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203a, CD203c, CD204, CD205, CD206, CD207, CD208, CD209, CD210, CDw210b, CD212, CD213a1, CD213a2, CD214, CD215, CD217, CD218a, CD218b, CD220, CD221, CD222, CD223, CD224, CD225, CD227, CD228, CD229, CD230, CD231, CD232, CD233, CD234, CD235a, CD235b, CD236, CD238, CD239, CD240CE, CD240D, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300b, CD300c, CD300d, CD300e, CD300f, CD300g, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD360, CD361, CD362, and CD363.

Illustrative tumor differentiation antigens are suitably selected from: α-fetoprotein (AFP), carcinoembryonic antigen (CEA) (e.g., CEACAM5, CEACAM6), CA-125, mucins (e.g., MUC1, MUC2, MUC3, MUC4, MUC5ac, MUC13, MUC16, etc.), epithelial tumor antigen (ETA), colon-specific antigen-p (CSA-p), tyrosinase, prostate-specific membrane antigen (PSMA), A33-antigen, transferrin receptor, tenascin, CA-IX and melanoma associated antigen.

Suitably, the hormone receptor is selected from estrogen receptor and progesterone receptor.

In some embodiments, the cell surface receptor is an integrin, illustrative examples of which include integrins α1, α2, α3, α4, α5, α6, α7, α8, α9, αD, αL, αM, αV, αX, αIIb, αIELb, β1, β2, β3, β4, β5, β6, β7, β8, α1β1, α2β1, α3β1, α4β1, α5β1, α6β1, α7β1, α8β1, α9β1, α4β7, α6β4, αDβ2, α1β2, αMβ2, αVβ1, αVβ3, αVβ5, αVβ6, αVβ8, αXβ2, αIIbβ3, and αIELbβ7.

Other cell surface receptors contemplated by the present invention include MAC-1 (β2 and cd11b), opioid receptors (μ and κ), FC receptors, serotonin receptors (5-HT, 5-HT6, 5-HT7), β-adrenergic receptors, leptin receptor, statin receptors, FAS receptor, BAFF receptor, FLT3 receptor and fibronectin receptor.

In preferred embodiments, the cell surface receptor elements are selected from members of the HER family, suitably HER1 and HER2. In other preferred embodiments, the cell surface receptor element is a programmed cell death protein (e.g. PD-1, also known as CD279, PD-2, PD-3, PD-4, PD-5, PD-6, etc.) or a steroid receptor (e.g., G-protein coupled receptor 30 (GPR30), ion channels such as GABA_{A} receptor, NMDA receptor and sigma receptors).

### 3.2 Clustering Analysis Methods

There are a number of widely available assays to detect clustering of cell surface receptor elements on a cell. For example, receptor ligands and/or receptor-specific antibodies can be labeled, and these labels detected to visualize clustering of receptor elements. In one example of this type of assay, a cell comprising the receptor element of interest is contacted with a receptor-specific antibody, and a fluorescently labeled secondary antibody that binds to the receptor-specific antibody. Using confocal scanning laser microscopy, fluorescence emitted from the secondary antibody can be detected to identify the location of the receptor elements. (Van Steensel, et al., 1995. J. Cell Sci 108: 3003-3011). In another example, a cell comprising a cell surface receptor element is contacted with a labeled ligand of the cell surface receptor and cell surface receptor element clustering analyzed by super resolution microscopy, as described for example by Kaufmann et al. (2011. J Microsc. 242(1): 46-54), Huber et al. (2011. PLoS One. 7(9):e44776), Wang et al. (2014. Biochim Biophys Acta. 1838(4): 1191-1198), Sams et al. (2014. J Biomed Opt. 19(1):011021), and in Example 7 below.

Alternatively, cell surface receptor clustering is analyzed by in situ proximity assay as described for example by Bellucci et al. (2014. Methods Mol Biol. 1174:397-405), Barros et al. (2014. Breast Cancer Res Treat. 144(2):273-85) and Pacchiana et al. (2014. Histochem Cell Biol. 142(5):593-60).

In other embodiments, FRET and FRAP microscopy can be employed to analyze receptor clustering, as described for example by Wallrabe et al. (2003. Biophys J. 85(1): 559-571), Wallrabe et al. (2003. J Biomed Opt. 8(3): 339-346) and de Heus et al. (2013. Methods Cell Biol. 117:305-321.

Other methods of receptor clustering analysis include: image correlation spectroscopy as described for example by Petersen et al. (1998. Faraday Discuss. (111):289-305), Kozer et al. (2013. Mol Biosyst. 9(7): 1849-1863), and Ciccotosto et al. (2013. Biophys J. 104(5): 1056-1064); electric field analysis, as described for example by Giugni et al. (1987. J Cell Biol. 104(5): 1291-1297), and Zhang et al. (2011. PLoS One. 6(10):e26805), electron microscopy, as described for example by Plowman et al. (2005. Proc Natl Acad Sci USA. 102(43): 15500-15505), and D'Amico et al. (2008. Micron. 39(1): 1-6); electron cryotomography, as described for example by Gold et al. (2014. Nat Commun. 5:4129); nanoparticle (NP) immunolabeling in combination with plasmon coupling microscopy (PCM), as described for example by Wang et al. (2012. Nano Lett. 12(6): 3231-3237) and Rong et al. (2012. PLoS One. 7(3):e34175); enzyme-mediated activation of radical source (EMARS) analysis, as described for example by Miyagawa-Yamaguchi et al. (2014. PLoS One. 9(3):e93054) and Kotani et al. (2008. Proc Natl Acad Sci USA. 105(21):7405-7409); and quantum dots analysis, as described for example by Li et al. (2010. Biophys J. 98(11): 2554-2563).

In other embodiments, a flow cytometer equipped with a doublet discriminator is used to determine the distribution of a labeled receptor ligand on a single cell. The distribution of the label on the cell is then correlated with the formation of receptor clusters to thereby determine the receptor element clustering status of the cell. An exemplary method of this type is disclosed in U.S. Pat. Appl. Pub. 2010/0018492.

Numerous ligands with specificity for cell surface receptors encompassed by the present invention are known, which can be used for clustering analysis. Many of these are also useful as therapeutic agents in accordance with the present invention. For example, any suitable antibody that binds a cell surface receptor of a tumor cell is contemplated for use in the practice of the present invention. Non-limiting examples of such antibodies are listed in Table 1.

TABLE 1

| | Trade | | | | |
|---|---|---|---|---|---|
| Name | name | Type | Source | Target | Use |
| 3F3 | | mAb | mouse | GD2 | neuroblastoma |
| 8H9 | | mAb | Mouse | B7-H3 | neuroblastoma, |
| | | | | | sarcoma, metastatic |
| | | | | | brain cancers |
| Abagovomab | | mAb | mouse | CA-125 | ovarian cancer |
| | | | | (imitation) | |
| Adecatumumab | | mAb | human | EpCAM | prostate and breast |
| | | | | | cancer |
| Aducanumab | | mAb | human | beta-amyloid | Alzheimer’s disease |
| Afutuzumab | | mAb | humanized | CD20 | lymphoma |
| Alacizumab pegol | | F(ab′) | humanized | VEGFR2 | cancer |
| | | 2 | | | |
| ALD518 | | ? | humanized | IL-6 | rheumatoid arthritis |
| Alemtuzumab | Campath, | mAb | humanized | CD52 | Multiple sclerosis |
| | MabCampath | | | | |
| Alirocumab | | mAb | human | NARP-1 | hypercholesterolemia |
| Altumomab | Hybri- | mAb | mouse | CEA | colorectal cancer |
| pentetate | ceaker | | | | (diagnosis) |
| Amatuximab | | mAb | chimeric | mesothelin | cancer |
| Anatumomab | | Fab | mouse | TAG-72 | non-small cell lung |
| mafenatox | | | | | carcinoma |
| Anifrolumab | | mAb | human | interferon α/β | systemic lupus |
| | | | | receptor | erythematosus |
| Anrukinzumab | | mAb | humanized | IL-13 | ? |
| (IMA-638) | | | | | |
| Apolizumab | | mAb | humanized | HLA-DR ? | hematological |
| | | | | | cancers |
| Arcitumomab | CEA-Scan | Fab′ | mouse | CEA | gastrointestinal |
| | | | | | cancers (diagnosis) |
| Atinumab | | mAb | human | RTN4 | ? |
| Atlizumab | Actemra, | mAb | humanized | IL-6 receptor | rheumatoid arthritis |
| (=tocilizumab) | RoActemra | | | | |
| Bavituximab | | mAb | chimeric | phosphatidylserine | cancer, viral |
| | | | | | infections |
| Bectumomab | LymphoScan | Fab′ | mouse | CD22 | non-Hodgkin’s |
| | | | | | lymphoma |
| | | | | | (detection) |
| Belimumab | Benlysta, | mAb | human | BAFF | non-Hodgkin |
| | LymphoStat- | | | | lymphoma etc. |
| | B | | | | |
| Besilesomab | Scintimun | mAb | mouse | CEA-related | inflammatory |
| | | | | antigen | lesions and |
| | | | | | metastases |
| | | | | | (detection) |
| Bevacizumab | Avastin | mAb | humanized | VEGF-A | metastatic cancer, |
| | | | | | retinopathy of |
| | | | | | prematurity |
| Bimagrumab | | mAb | human | ACVR2B | myostatin inhibitor |
| Bivatuzumab | | mAb | humanized | CD44 v6 | squamous cell |
| mertansine | | | | | carcinoma |
| Blinatumomab | | BiTE | mouse | CD19 | cancer |
| Brentuximab vedotin | | mAb | chimeric | CD30 | hematologic cancers |
| | | | | (TNFRSF8) | |
| Cantuzumab | | mAb | humanized | mucin CanAg | colorectal cancer |
| mertansine | | | | | etc. |
| Cantuzumab | | mAb | humanized | MUC1 | cancers |
| ravtansine | | | | | |
| Caplacizumab | | mAb | humanized | VWF | ? |
| Capromab pendetide | Prostascint | mAb | mouse | prostatic | prostate cancer |
| | | | | carcinoma cells | (detection) |
| Carlumab | | mAb | human | MCP-1 | oncology/immune |
| | | | | | indications |
| Catumaxomab | Removab | 3funct | rat/mouse | EpCAM, CD3 | ovarian cancer, |
| | | | hybrid | | malignant ascites, |
| | | | | | gastric cancer |
| cBR96-doxorubicin | | mAb | humanized | Lewis-Y antigen | cancer |
| immunoconjugate | | | | | |
| CC49 | | mAb | mouse | TAG-72 | tumor detection |
| Certolizumab pegol | Cimzia | Fab′ | humanized | TNF-α | Crohn’s disease |
| Cetuximab | Erbitux | mAb | chimeric | EGFR | metastatic |
| | | | | | colorectal cancer |
| | | | | | and head and neck |
| | | | | | cancer |
| Ch.14.18 | | mAb | chimeric | ??? | neuroblastoma |
| Citatuzumab bogatox | | Fab | humanized | EpCAM | ovarian cancer and |
| | | | | | other solid tumors |
| Cixutumumab | | mAb | human | IGF-1 receptor | solid tumors |
| Clazakizumab | | mAb | humanized | Oryctolagus | rheumatoid arthritis |
| | | | | cuniculus | |
| Clenoliximab | | mAb | chimeric | CD4 | rheumatoid arthritis |
| Clivatuzumab | hPAM4- | mAb | humanized | MUC1 | pancreatic cancer |
| tetraxetan | Cide | | | | |
| Conatumumab | | mAb | human | TRAIL-R2 | cancer |
| Concizumab | | mAb | humanized | TFPI | bleeding |
| Dacetuzumab | | mAb | humanized | CD40 | hematologic cancers |
| Dalotuzumab | | mAb | humanized | insulin-like | cancer etc. |
| | | | | growth factor I | |
| | | | | receptor | |
| Daratumumab | | mAb | human | CD38 (cyclic | ? |
| | | | | ADP ribose | |
| | | | | hydrolase) | |
| Demcizumab | | mAb | humanized | DLL4 | cancer |
| Denosumab | Prolia | mAb | human | RANKL | osteoporosis, bone |
| | | | | | metastases etc. |
| Detumomab | | mAb | mouse | B-lymphoma | lymphoma |
| | | | | cell | |
| Dinutuximab | | mAb | chimeric | ganglioside GD2 | neuroblastoma |
| Dorlimomab aritox | | F(ab′) | mouse | ? | ? |
| | | 2 | | | |
| Drozitumab | | mAb | human | DR5 | cancer etc. |
| Duligotumab | | mAb | human | HER3 | ? |
| Durvalumab | | mAb | human | [CD274]] | cancer |
| Dusigitumab | | mAb | human | ILGF2 | cancer |
| Ecromeximab | | mAb | chimeric | GD3 ganglioside | malignant |
| | | | | | melanoma |
| Edrecolomab | Panorex | mAb | mouse | EpCAM | colorectal carcinoma |
| Elotuzumab | | mAb | humanized | SLAMF7 | multiple myeloma |
| Elsilimomab | | mAb | mouse | IL-6 | ? |
| Emibetuzumab | | mAb | humanized | HHGFR | cancer |
| Enavatuzumab | | mAb | humanized | TWEAK receptor | cancer etc. |
| Enfortumab vedotin | | mAb | human | AGS-22M6 | cancer expressing |
| | | | | | Nectin-4 |
| Enlimomab pegol | | mAb | mouse | ICAM-1 (CD54) | ? |
| Enoticumab | | mAb | human | DLL4 | ? |
| Ensituximab | | mAb | chimeric | 5AC | cancer |
| Epitumomab | | mAb | mouse | episialin | ? |
| cituxetan | | | | | |
| Epratuzumab | | mAb | humanized | CD22 | cancer, SLE |
| Ertumaxomab | Rexomun | 3funct | rat/mouse | HER2/neu, CD3 | breast cancer etc. |
| | | | hybrid | | |
| Etaracizumab | Abegrin | mAb | humanized | integrin ανβ3 | melanoma, prostate |
| | | | | | cancer, ovarian |
| | | | | | cancer etc. |
| Faralimomab | | mAb | mouse | interferon | ? |
| | | | | receptor | |
| Farletuzumab | | mAb | humanized | folate receptor | ovarian cancer |
| | | | | 1 | |
| Fasinumab | | mAb | human | HNGF | ? |
| FBTA05 | Lymphomun | 3funct | rat/mouse | CD20 | chronic lymphocytic |
| | | | hybrid | | leukaemia |
| Ficlatuzumab | | mAb | humanized | HGF | cancer etc. |
| Figitumumab | | mAb | human | IGF-1 receptor | adrenocortical |
| | | | | | carcinoma, non- |
| | | | | | small cell lung |
| | | | | | carcinoma etc. |
| Flanvotumab | | mAb | human | TYRP1 | melanoma |
| | | | | (glycoprotein | |
| | | | | 75) | |
| Foralumab | | mAb | human | CD3 epsilon | ? |
| Fresolimumab | | mAb | human | TGF-β | idiopathic |
| | | | | | pulmonary fibrosis, |
| | | | | | focal segmental |
| | | | | | glomerulosclerosis, |
| | | | | | cancer |
| Futuximab | | mAb | chimeric | EGFR | ? |
| Galiximab | | mAb | chimeric | CD80 | B-cell lymphoma |
| Ganitumab | | mAb | human | IGF-I | cancer |
| Gemtuzumab | Mylotarg | mAb | humanized | CD33 | acute myelogenous |
| ozogamicin | | | | | leukemia |
| Girentuximab | Rencarex | mAb | chimeric | carbonic | clear cell renal cell |
| | | | | anhydrase 9 | carcinoma[73] |
| | | | | (CA-IX) | |
| Glembatumumab | | mAb | human | GPNMB | melanoma, breast |
| vedotin | | | | | cancer |
| Ibritumomab | Zevalin | mAb | mouse | CD20 | non-Hodgkin’s |
| tiuxetan | | | | | lymphoma |
| Icrucumab | | mAb | human | VEGFR-1 | cancer etc. |
| Igovomab | Indimacis- | F(ab′) | mouse | CA-125 | ovarian cancer |
| | 125 | 2 | | | (diagnosis) |
| IMAB362 | | mAb | human | CLDN18.2 | gastrointestinal |
| | | | | | adenocarcinomas |
| | | | | | and pancreatic |
| | | | | | tumor |
| Imgatuzumab | | mAb | humanized | EGFR | cancer |
| Inclacumab | | mAb | human | selectin P | ? |
| Indatuximab | | mAb | chimeric | SDC1 | cancer |
| ravtansine | | | | | |
| Inotuzumab | | mAb | humanized | CD22 | cancer |
| ozogamicin | | | | | |
| Intetumumab | | mAb | human | CD51 | solid tumors |
| | | | | | (prostate cancer, |
| | | | | | melanoma) |
| Ipilimumab | Yervoy | mAb | human | CD152 | melanoma |
| Iratumumab | | mAb | human | CD30 | Hodgkin’s |
| | | | | (TNFRSF8) | lymphoma |
| Itolizumab | | mAb | humanized | CD6 | ? |
| Ixekizumab | | mAb | humanized | IL-17A | autoimmune |
| | | | | | diseases |
| Labetuzumab | CEA-Cide | mAb | humanized | CEA | colorectal cancer |
| Lambrolizumab | | mAb | humanized | PDCD1 | antineoplastic agent |
| Lexatumumab | | mAb | human | TRAIL-R2 | cancer |
| Lifastuzumab vedotin | | mAb | humanized | phosphate- | cancer |
| | | | | sodium co- | |
| | | | | transporter | |
| Lintuzumab | | mAb | humanized | CD33 | cancer |
| Lirilumab | | mAb | human | KIR2D | ? |
| Lorvotuzumab | | mAb | humanized | CD56 | cancer |
| mertansine | | | | | |
| Lucatumumab | | mAb | human | CD40 | multiple myeloma, |
| | | | | | non-Hodgkin’s |
| | | | | | lymphoma, |
| | | | | | Hodgkin’s |
| | | | | | lymphoma |
| Lumiliximab | | mAb | chimeric | CD23 (IgE | chronic lymphocytic |
| | | | | receptor) | leukemia |
| Mapatumumab | | mAb | human | TRAIL-R1 | cancer |
| Margetuximab | | mAb | humanized | ch4D5 | cancer |
| Maslimomab | | ? | mouse | T-cell receptor | ? |
| Matuzumab | | mAb | humanized | EGFR | colorectal, lung and |
| | | | | | stomach cancer |
| Milatuzumab | | mAb | humanized | CD74 | multiple myeloma |
| | | | | | and other |
| | | | | | hematological |
| | | | | | malignancies |
| Minretumomab | | mAb | mouse | TAG-72 | ? |
| Mitumomab | | mAb | mouse | GD3 ganglioside | small cell lung |
| | | | | | carcinoma |
| Mogamulizumab | | mAb | humanized | CCR4 | cancer |
| Morolimumab | | mAb | human | Rhesus factor | ? |
| Moxetumomab | | mAb | mouse | CD22 | cancer |
| pasudotox | | | | | |
| Nacolomab tafenatox | | Fab | mouse | C242 antigen | colorectal cancer |
| Namilumab | | mAb | human | CSF2 | ? |
| Naptumomab | | Fab | mouse | 5T4 | non-small cell lung |
| estafenatox | | | | | carcinoma, renal |
| | | | | | cell carcinoma |
| Narnatumab | | mAb | human | RON | cancer |
| Necitumumab | | mAb | human | EGFR | non-small cell lung |
| | | | | | carcinoma |
| Nerelimomab | | mAb | mouse | TNF-α | ? |
| Nesvacumab | | mAb | human | angiopoietin 2 | cancer |
| Nimotuzumab | Theracim, | mAb | humanized | EGFR | squamous cell |
| | Theraloc | | | | carcinoma, head |
| | | | | | and neck cancer, |
| | | | | | nasopharyngeal |
| | | | | | cancer, glioma |
| Nivolumab | | mAb | human | IgG4 | cancer |
| Nofetumomab | Verluma | Fab | mouse | ? | cancer (diagnosis) |
| merpentan | | | | | |
| Ocaratuzumab | | mAb | humanized | CD20 | cancer |
| Ofatumumab | Arzerra | mAb | human | CD20 | chronic lymphocytic |
| | | | | | leukemia etc. |
| Olaratumab | | mAb | human | PDGF-R α | cancer |
| Olokizumab | | mAb | humanized | IL6 | ? |
| Onartuzumab | | mAb | humanized | human scatter | cancer |
| | | | | factor receptor | |
| | | | | kinase | |
| Ontuxizumab | | mAb | chimeric/hu- | TEM1 | cancer |
| | | | manized | | |
| Oportuzumab | | scFv | humanized | EpCAM | cancer |
| monatox | | | | | |
| Oregovomab | OvaRex | mAb | mouse | CA-125 | ovarian cancer |
| Orticumab | | mAb | human | oxLDL | ? |
| Otlertuzumab | | mAb | humanized | CD37 | cancer |
| Panitumumab | Vectibix | mAb | human | EGFR | colorectal cancer |
| Pankomab | | mAb | humanized | tumor specific | ovarian cancer |
| | | | | glycosylation of | |
| | | | | MUC1 | |
| Parsatuzumab | | mAb | human | EGFL7 | cancer |
| Pateclizumab | | mAb | humanized | LTA | TNF |
| Patritumab | | mAb | human | HER3 | cancer |
| Pembrolizumab | Keytruda | mAb | humanized | PDCD1 | cancer etc. |
| Pemtumomab | Theragyn | ? | mouse | MUC1 | cancer |
| Pertuzumab | Omnitarg | mAb | humanized | HER2/neu | cancer |
| Pidilizumab | | mAb | humanized | PD-1 | cancer and |
| | | | | | infectious diseases |
| Pinatuzumab vedotin | | mAb | humanized | CD22 | cancer |
| Pintumomab | | mAb | mouse | adenocarcinoma | adenocarcinoma |
| | | | | antigen | (imaging) |
| Placulumab | | mAb | human | human TNF | ? |
| Polatuzumab vedotin | | mAb | humanized | CD79B | cancer |
| Pritumumab | | mAb | human | vimentin | brain cancer |
| Quilizumab | | mAb | humanized | IGHE | ? |
| Racotumomab | | mAb | mouse | N- | cancer |
| | | | | glycolylneuraminic | |
| | | | | acid | |
| Radretumab | | mAb | human | fibronectin | cancer |
| | | | | extra domain-B | |
| Ramucirumab | | mAb | human | VEGFR2 | solid tumors |
| Rilotumumab | | mAb | human | HGF | solid tumors |
| Rituximab | MabThera, | mAb | chimeric | CD20 | lymphomas, |
| | Rituxan | | | | leukemias, some |
| | | | | | autoimmune |
| | | | | | disorders |
| Robatumumab | | mAb | human | IGF-1 receptor | cancer |
| Roledumab | | mAb | human | RHD | ? |
| Samalizumab | | mAb | humanized | CD200 | cancer |
| Satumomab | | mAb | mouse | TAG-72 | cancer (diagnosis) |
| pendetide | | | | | |
| Seribantumab | | mAb | human | ERBB3 | cancer |
| Setoxaximab | | mAb | chimeric | E. coli shiga | ? |
| | | | | toxin type-2 | |
| SGN-CD19A | | mAb | humanized | CD19 | acute lymphoblastic |
| | | | | | leukemia and B-cell |
| | | | | | non-Hodgkin |
| | | | | | lymphoma |
| SGN-CD33A | | mAb | humanized | CD33 | Acute myeloid |
| | | | | | leukemia |
| Sibrotuzumab | | mAb | humanized | FAP | cancer |
| Siltuximab | | mAb | chimeric | IL-6 | cancer |
| Sofituzumab vedotin | | CA | ovarian | | |
| | | 125 | cancer | | |
| Solitomab | | mAb | mouse | EpCAM | ? |
| Sontuzumab | | mAb | humanized | episialin | ? |
| Tabalumab | | mAb | human | BAFF | B-cell cancers |
| Tacatuzumab | AFP-Cide | mAb | humanized | alpha- | cancer |
| tetraxetan | | | | fetoprotein | |
| Taplitumomab | | mAb | mouse | CD19 | cancer |
| paptox | | | | | |
| Tarextumab | | mAb | human | Notch receptor | cancer |
| Telimomab aritox | | Fab | mouse | ? | ? |
| Tenatumomab | | mAb | mouse | tenascin C | cancer |
| Teneliximab | | mAb | chimeric | CD40 | ? |
| Teprotumumab | | mAb | human | CD221 | hematologic tumors |
| TGN1412 | | ? | humanized | CD28 | chronic lymphocytic |
| | | | | | leukemia, |
| | | | | | rheumatoid arthritis |
| Ticilimumab | | mAb | human | CTLA-4 | cancer |
| (tremelimumab) | | | | | |
| Tigatuzumab | | mAb | humanized | TRAIL-R2 | cancer |
| Tildrakizumab | | mAb | humanized | IL23 | immunologically |
| | | | | | mediated |
| | | | | | inflammatory |
| | | | | | disorders |
| TNX-650 | | ? | humanized | IL-13 | Hodgkin’s |
| | | | | | lymphoma |
| Tositumomab | Bexxar | ? | mouse | CD20 | follicular lymphoma |
| Tovetumab | | mAb | human | CD140a | cancer |
| Trastuzumab | Herceptin | mAb | humanized | HER2/neu | breast cancer |
| TRBS07 | Ektomab | 3funct | ? | GD2 | melanoma |
| Tregalizumab | | mAb | humanized | CD4 | ? |
| Tremelimumab | | mAb | human | CTLA-4 | cancer |
| Tucotuzumab | | mAb | humanized | EpCAM | cancer |
| celmoleukin | | | | | |
| Ublituximab | | mAb | chimeric | MS4A1 | cancer |
| Urelumab | | mAb | human | 4-1BB | cancer etc. |
| Vantictumab | | mAb | human | Frizzled | cancer |
| | | | | receptor | |
| Vapaliximab | | mAb | chimeric | AOC3 (VAP-1) | ? |
| Varlilumab | | mAb | human | CD27 | ? |
| Vatelizumab | | mAb | humanized | ITGA2 | ? |
| Veltuzumab | | mAb | humanized | CD20 | non-Hodgkin’s |
| | | | | | lymphoma |
| Vepalimomab | | mAb | mouse | AOC3 (VAP-1) | inflammation |
| Vesencumab | | mAb | human | NRP1 | ? |
| Volociximab | | mAb | chimeric | integrin α5β1 | solid tumors |
| Vorsetuzumab | | mAb | humanized | CD70 | cancer |
| mafodotin | | | | | |
| Votumumab | HumaSPECT | mAb | human | tumor antigen | colorectal tumors |
| | | | | CTAA16.88 | |
| Zalutumumab | HuMax- | mAb | human | EGFR | squamous cell |
| | EGFr | | | | carcinoma of the |
| | | | | | head and neck |
| Zatuximab | | mAb | chimeric | HER1 | cancer |
| Ziralimumab | | mAb | human | CD147 (basigin) | ? |

Other therapeutic antibodies Include anti-EGFR antibodies such as but not limited to include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or cetuximab; ERBITUX™) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or panitumumab (see, WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-α for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al. (2004) J. Biol. Chem. 279(29): 30375-30384); anti-HER2 antibodies, illustrative examples of which include HERCEPTIN™ (trastuzumab) (Genentech, Calif.); PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); VITAXIN™ which is a humanized anti-αVβ3 integrin antibody (Applied Molecular Evolution/Medimmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ (rituximab) which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); ZEVALIN™ is a radiolabeled murine anti-CD20 antibody (IDEC/Schering AG); and IDEC-152 (lumiliximab) is a primatized anti-CD23 antibody (IDEC/Seikagaku).

In specific embodiments, the antibody comprises an Fc region of an immunoglobulin. Alternatively, or in addition, the antibody is a multivalent (e.g., bivalent) antibody.

Numerous ligands are also available for binding to their cognate surface receptors including ligands of the following classes: protein, small organic molecule, carbohydrates (including polysaccharides), polynucleotide, lipids, etc. Representative examples of such ligands include EGFR ligand, EGF, TGF-alpha, TGF-α, amphiregulin, heparin-binding EGF-like growth factor, betacellulin, and epiregulin, interferon, interferon gamma, interferon beta, interferon alpha, interleukin-1, interleukin-2, interleukin-4, interleukin-6, interleukin-8, interleukin-10, interleukin-12, tumor necrosis factor, tumor necrosis factor alpha, tumor necrosis factor beta, hepatocyte growth factor, vascular endothelial growth factor (VEGF), fibroblast growth factor (FGF), platelet derived growth factor (PDGF), Flt3 ligand, insulin, insulin-like growth factor, growth hormone, nerve growth factor, brain-derived neurotrophic factor, enzymes, endostatin, angiostatin, thrombospondin, urokinase, streptokinase, granulocyte-macrophage colony-stimulating factor (GM-CSF), granulocyte colony-stimulating factor (G-CSF), Toll-like receptor, melanoma stimulating hormone (MSH), thrombopoietin, calcitonin, parathyroid hormone (PTH) and its fragments, protease inhibitors, adrenocorticotropin, gonadotropin releasing hormone, follicle stimulating hormone, thrombopoietin, filgrastim, prostaglandins, epoprostenol, prostacyclin, cyclosporine, vasoactive intestinal peptide (VIP), vancomycin, antimicrobials, polymyxin b, anti-fungal agents, anti-viral agents, enfuvirtide, doxorubicin, etoposide, fentanyl, ketamine, and vitamins.

In advantageous embodiments, the cell surface receptor element clustering analysis assays used for the tumor classification methods of the present invention are those described in the examples.

### 4. Methods for Stratifying Responders and Non-Responders and Uses Therefor

The tumor classification methods disclosed herein are useful for stratifying cancer-affected subjects into responders and non-responders to a therapeutic agent that is a ligand of a cell surface receptor, wherein the cancer comprises tumors that are positive for cell surface receptor elements. In accordance with the present invention, when a subject's tumor is determined as having unimpaired clustering of the cell surface receptor elements, the subject is stratified as a responder to the therapeutic agent. Conversely, when a subject's tumor is determined as having impaired clustering of the cell surface receptor elements, the subject is stratified as a non-responder to the therapeutic agent. This stratification, in turn, permits better management of cancer-affected subjects in which responders are administered the therapeutic agent and non-responders are offered an alternate treatment.

Thus, subjects identified as responders are administered the therapeutic agent, illustrative examples of which are listed above.

Conversely, subjects identified as non-responders are administered an alternative therapy to treat the cancer, non-limiting examples of which include radiotherapy, surgery, chemotherapy, hormone ablation therapy, pro-apoptosis therapy and immunotherapy.

### 4.1 Radiotherapy

Radiotherapies include radiation and waves that induce DNA damage for example, γ-irradiation, X-rays, UV irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half life of the isotope, the strength and type of radiation emitted, and the uptake by the neoplastic cells.

Non-limiting examples of radiotherapies include conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine.

### 4.2 Chemotherapy

Chemotherapeutic agents may be selected from any one or more of the following categories:
(i) antiproliferative/antineoplastic drugs and combinations thereof, as used in medical oncology, such as alkylating agents (e.g., cis-platin, carboplatin, cyclophosphamide, nitrogen mustard, melphalan, chlorambucil, busulphan and nitrosoureas); antimetabolites (e.g., antifolates such as fluoropyridines like 5-fluorouracil and tegafur, raltitrexed, methotrexate, cytosine arabinoside and hydroxyurea; anti-tumor antibiotics (e.g., anthracyclines like adriamycin, bleomycin, doxorubicin, daunomycin, epirubicin, idarubicin, mitomycin-C, dactinomycin and mithramycin); antimitotic agents (e.g., vinca alkaloids like vincristine, vinblastine, vindesine and vinorelbine and taxoids like paclitaxel and docetaxel; and topoisomerase inhibitors (e.g., epipodophyllotoxins like etoposide and teniposide, amsacrine, topotecan and camptothecin);
(ii) cytostatic agents such as antioestrogens (e.g., tamoxifen, toremifene, raloxifene, droloxifene and iodoxyfene), estrogen receptor down regulators (for example fulvestrant), antiandrogens (e.g., bicalutamide, flutamide, nilutamide and cyproterone acetate), UH antagonists or LHRH agonists (e.g., goserelin, leuprorelin and buserelin), progestogens (e.g., megestrol acetate), aromatase inhibitors (e.g., as anastrozole, letrozole, vorazole and exemestane) and inhibitors of 5α-reductase such as finasteride;
(iii) agents which inhibit cancer cell invasion (e.g., metalloproteinase inhibitors like marimastat and inhibitors of urokinase plasminogen activator receptor function);
(iv) inhibitors of growth factor function, for example such inhibitors include growth factor antibodies, growth factor receptor antibodies (e.g., the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazoli-n-4-amine (CI 1033)), for example inhibitors of the platelet-derived growth factor family and for example inhibitors of the hepatocyte growth factor family;
(v) anti-angiogenic agents such as those which inhibit the effects of vascular endothelial growth factor, (e.g., the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (e.g., linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense; and
(viii) gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

### 4.3 Immunotherapy

Immunotherapy approaches, include for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

### 4.4 Other Therapies

Examples of other cancer therapies include phototherapy, cryotherapy, toxin therapy or pro-apoptosis therapy. One of skill in the art would know that this list is not exhaustive of the types of treatment modalities available for cancer and other hyperplastic lesions.

Generally, the therapeutic agents described above are administered in the form of pharmaceutical compositions that optionally comprise a pharmaceutically acceptable carrier, excipient and/or stabilizer (Remington's Pharmaceutical Sciences 16th edition, Osol, A. Ed. (1980)). These compositions are generally in the form of lyophilized formulations or aqueous solutions. Antibody crystals are also contemplated (see, U.S. Pat. Appl. 2002/0136719). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized antibody formulations are described in WO 97/04801.

The pharmaceutical compositions may also contain more than one active compound as necessary for the particular indication being treated, desirably those with complementary activities that do not adversely affect each other. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

### 5. Kits

As noted above, the present invention contemplates the use of clustering assays for determining the cell surface receptor element clustering status of a tumor, and/or for determining the therapeutic agent sensitivity of a tumor, and/or for stratifying a subject into a treatment subgroup selected from therapeutic agent responder and non-responder. All the essential materials and reagents (e.g., labels, antibodies, ligands etc.) required for these assays may be assembled together in a kit. The kits may also optionally include appropriate reagents for detection of labels, positive and negative controls, washing solutions, blotting membranes, microtiter plates dilution buffers and the like. Such kits also generally will comprise, in suitable means, distinct containers for each individual reagent. The kit can also feature various devices and reagents for performing one of the assays described herein; and/or printed instructions for using the kit for assaying receptor internalization.

In some embodiments, the methods described generally herein are performed, at least in part, by a processing system, such as a suitably programmed computer system. A stand-alone computer, with the microprocessor executing applications software allowing the above-described methods to be performed, may be used. Alternatively, the methods can be performed, at least in part, by one or more processing systems operating as part of a distributed architecture. For example, a processing system can be used to assay cell surface receptor element clustering. A processing system also can be used to determine the cell surface receptor element clustering status of a tumor, and to stratify a subject into a treatment subgroup selected from therapeutic agent responders and non-responders, on the basis of the cell surface receptor element clustering status. In some examples, commands inputted to the processing system by a user assist the processing system in making these determinations.

In one example, a processing system includes at least one microprocessor, a memory, an input/output device, such as a keyboard and/or display, and an external interface, interconnected via a bus. The external interface can be utilized for connecting the processing system to peripheral devices, such as a communications network, database, or storage devices. The microprocessor can execute instructions in the form of applications software stored in the memory to allow a process (e.g., determination of cell surface receptor element clustering status, and/or determination of therapeutic agent sensitivity of a tumor, and/or stratification of a subject into a treatment subgroup selected from therapeutic agent responder and non-responder) to be performed, as well as to perform any other required processes, such as communicating with the computer systems. The applications software may include one or more software modules, and may be executed in a suitable execution environment, such as an operating system environment, or the like.

In order that the invention may be readily understood and put into practical effect, particular preferred embodiments will now be described by way of the following non-limiting examples.

### EXAMPLES

### Example 1

### Trastuzumab can Mediate Anti-Proliferative Effects Against HER2 Overexpressing Breast Cancer Cell Lines

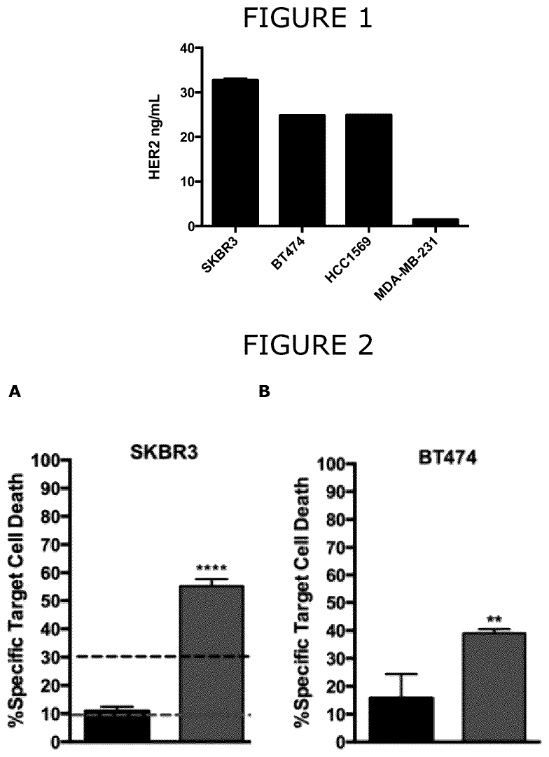

The level of HER2 expression of several breast cancer cell lines (SKBR3, BT474, HCC1569 and MDA-MB-231 cells) was assessed using an ELISA assay. HER2 was found to be over-expressed in SKBR3, moderately for BT474 and HCC1569 cells and negligibly for MDA-MB-231 cells (FIG. 1).

### Example 2

### Resistance to the Anti-Proliferative Effect of Trastuzumab does not Correlate with Resistance to its Other Mechanism of Action

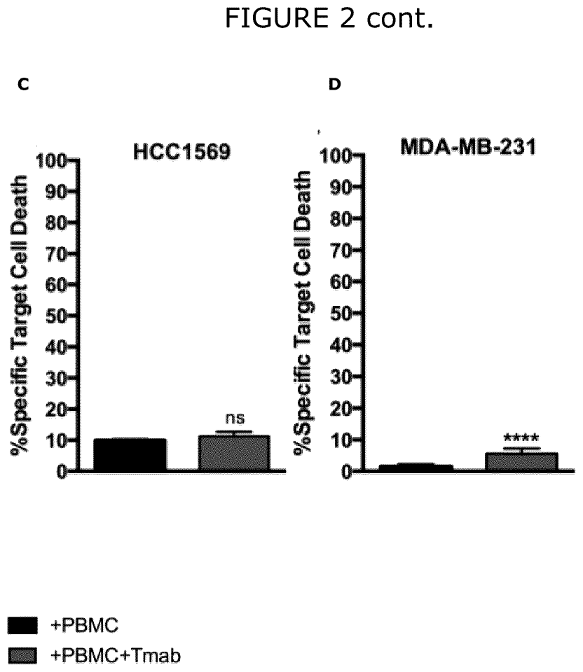

The sensitivity of the cell lines resistant to the anti-proliferative effects to Trastuzumab mediated cytotoxicity was also assessed. Amongst cell lines tested, resistance to the anti-proliferative effects of Trastuzumab did not necessarily confer resistance to immune-dependent cytotoxicity. SKBR3 cells were highly and significantly sensitive to Trastuzumab mediated cytotoxicity in the presence of PBMCs when compared with un-treated controls (target cells+PBMC) (FIG. 2A). BT474 cells were also sensitive to Trastuzumab-mediated immune-dependent cytotoxicity (FIG. 2B). However, HCC1569 cells, which express comparable levels of HER2 to BT474 (FIG. 1), were insensitive to Trastuzumab-mediated cytotoxicity, as they failed to demonstrate an increase in specific target cell death following treatment with Trastuzumab (FIG. 2C). This suggested that the HER2 expression level is not the only determinant of sensitivity to the immune-mediated cytotoxic effects of Trastuzumab. Moreover, the response of MDA-MB-231 cells, which was used as a HER2 negative control, was also tested in the immune dependent cytotoxicity assay to evaluate the contribution of mAb/target cell engagement to immune-dependent Trastuzumab mediated cytotoxic effects. Although the increase in specific cell death of MDA-MB-231 cells was significant following treatment with Trastuzumab, this increase did not reach any significant tumor cell killing levels (FIG. 2D), and these cells were thus considered insensitive to Trastuzumab treatment which is consistent with their low level of HER2 expression (FIG. 1).

### Example 3

### HER2 Cell Surface Level Correlates with Sensitivity of Cells to Trastuzumab Mediated Immune-Cell-Dependent Anti-Tumor Effect In Vitro

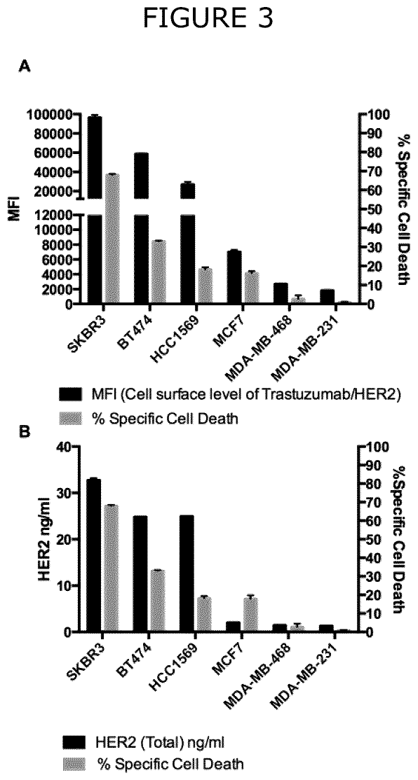

The relative level of HER2 displayed on the plasma membrane following a 4-hour Trastuzumab treatment was compared amongst SKBR3, BT474, HCC1569 and MDA-MB-231. To quantitate cell surface levels of Trastuzumab/HER2 following treatment with Trastuzumab, cells were labeled with a secondary antibody, capable of binding Trastuzumab only exposed at the cell surface, and the mean fluorescence intensity (MFI) of each cell line was determined by flow cytometry. A significant correlation (R=0.96; P=<0.001) was identified between the relative cell surface level of Trastuzumab/HER2 and relative specific cell death of cell lines treated with Trastuzumab in the immune-dependent cytotoxicity assays (FIG. 3A). By comparison, analysis of relative total HER2 expression and relative specific cell death of all cell lines revealed a substantially less significant correlation (R₂=0.64; P=<0.05) (FIG. 3B). This suggested that only Trastuzumab displayed at the cell surface of target may be capable of participating in immune dependent cytotoxicity.

### Example 4

### Blocking Dynamin-Mediated Endocytosis Enhances the Sensitivity of Breast Cancer Cell Lines to In Vitro Trastuzumab Mediated Immune-Dependent Cytotoxicity

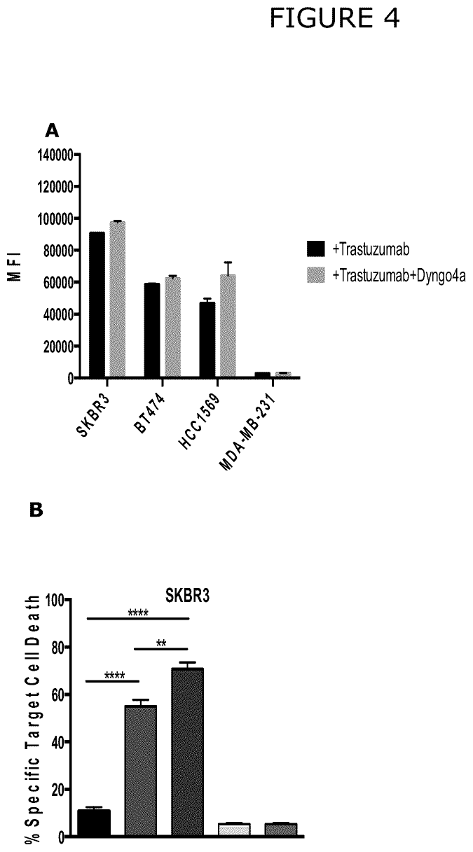

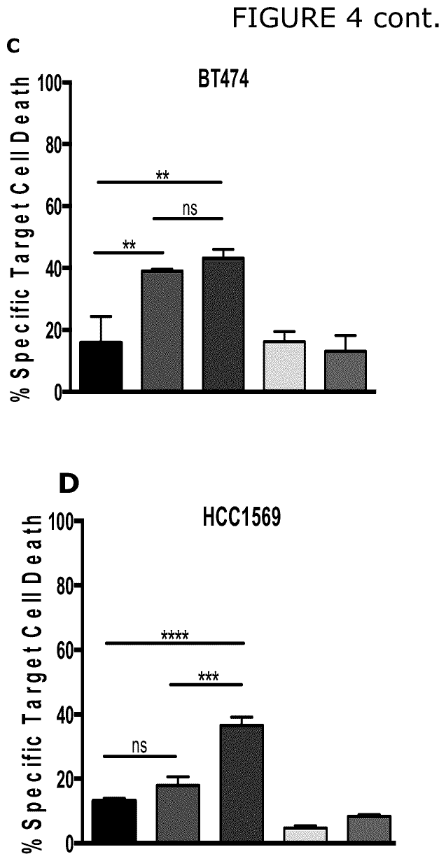

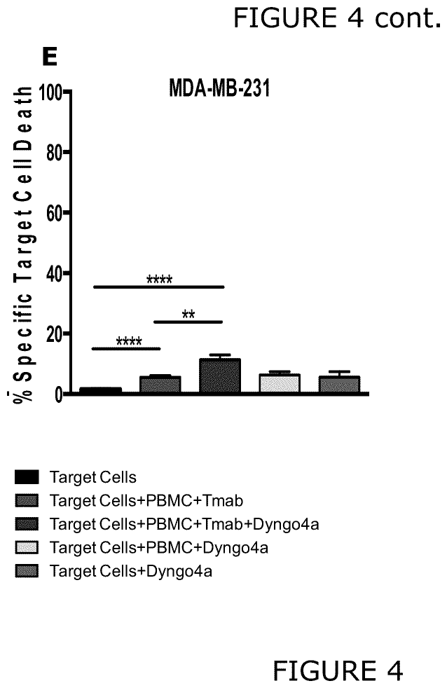

Following the finding that the cell surface level of Trastuzumab binding was a major determinant of sensitivity to the immune modulating effects of Trastuzumab, the effect of Trastuzumab/HER2 endocytosis inhibition on the mAb mediated immune-dependent cytotoxicity was examined. Dyngo4a, a small molecule inhibitor of dynamin, was used to block dynamin-mediated endocytosis (Mccluskey et al., 2013) and added into the immune-dependent cytotoxicity assays. It was found that treatment with Dyngo4a enhanced the level of Trastuzumab bound HER2 at the cell surface (FIG. 4A), which enhanced the sensitivity of cells to the immune-mediated cytotoxic effects of Trastuzumab (FIG. 4B-E). SKBR3, BT474, HCC1569 and MDA-MB-231 cells all demonstrated a relative increase in MFI indicating that the proportion of Trastuzumab bound at the cell surface had been increased following Dyngo4a treatment (FIG. 3A). When tested in the context of the immune-dependent cytotoxicity assays, SKBR3 (FIG. 3B), HCC1569 (FIG. 4D) and MDA-MB-231 (FIG. 4E) cells treated with Dyngo4a in combination with Trastuzumab were significantly more sensitive to Trastuzumab mediated cytotoxicity compared with the mAb single-agent treated control. However, the specific cell death of BT474 cells was not enhanced following treatment with Dyngo4a (FIG. 4C). As Dyngo4a was shown to be non-toxic, the elevation in specific cell death associated with the combination of Trastuzumab with Dyngo4a was most likely due to inhibition of HER2 endocytosis resulting in increased cell surface Trastuzumab and thus an increased in immune mediated cytotoxicity, and this was postulated to have resulted from a change in receptor clustering.

### Example 5

### Inhibition of Dynamin-Mediated Endocytosis in HER1 Overexpressing Breast Cancer Cell Lines Enhances Immune-Dependent Cytotoxicity Mediated by Cetuximab

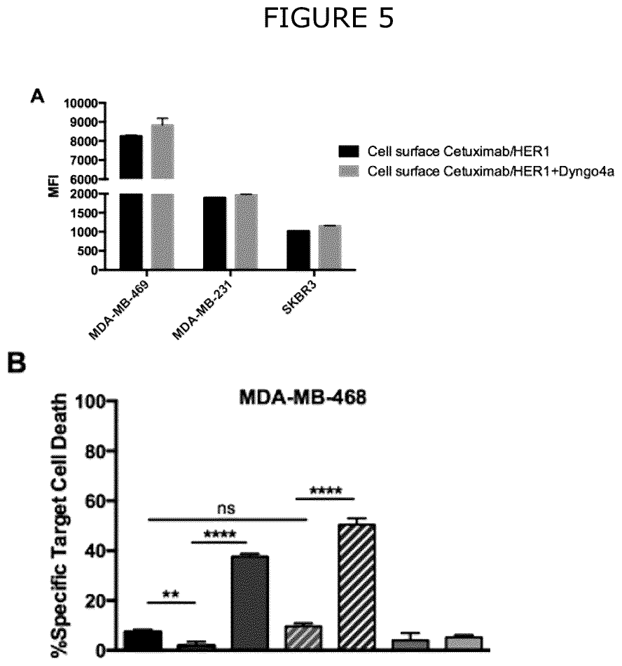

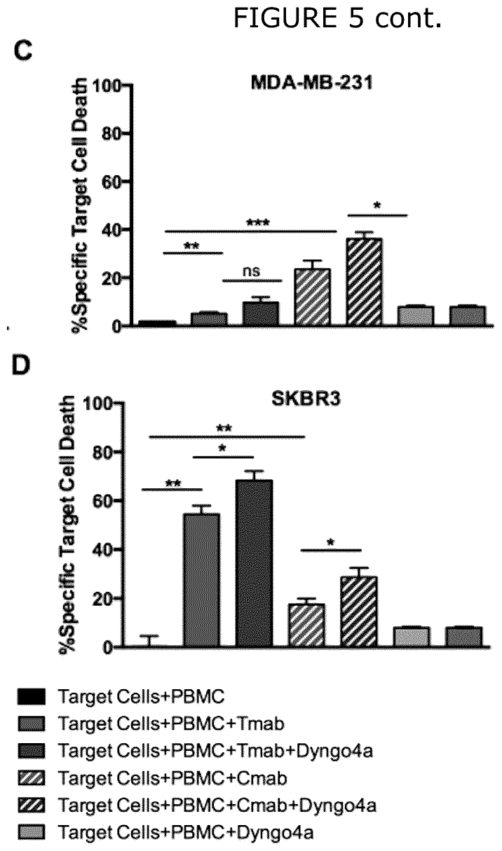

The level of Cetuximab/HER1 bound at the plasma membrane following a 4-hour treatment with Cetuximab or Cetuximab in combination with Dyngo4a was quantitated as a measure of MFI. It was revealed that the combination treatment increased the level of Cetuximab/HER1 at the cell surface (FIG. 5A). It was further demonstrated that treatment of MDA-MB-468, MDA-MB-231 and SKBR3 cells with Dyngo4a was sufficient to enhance the sensitivity of these cell lines to immune-dependent cytotoxicity of Cetuximab as well as of Trastuzumab, that was not due to toxicity associated with Dyngo4a treatment (FIGS. 58, 5C & 5D). Together, these data indicate that increasing the level of Cetuximab/HER1 displayed at the plasma membrane of Cetuximab treated cells can enhance immune effector cell mediated cytotoxicity.

### Example 6

### Antibody-Mediated Immune Dependent Cytotoxicity May be a FcrRIII Dependent, NK Cell Mediated Process

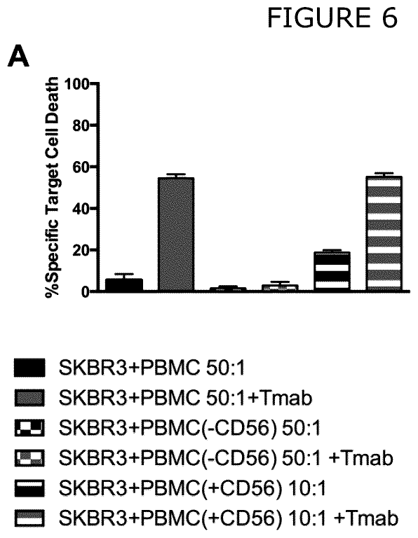

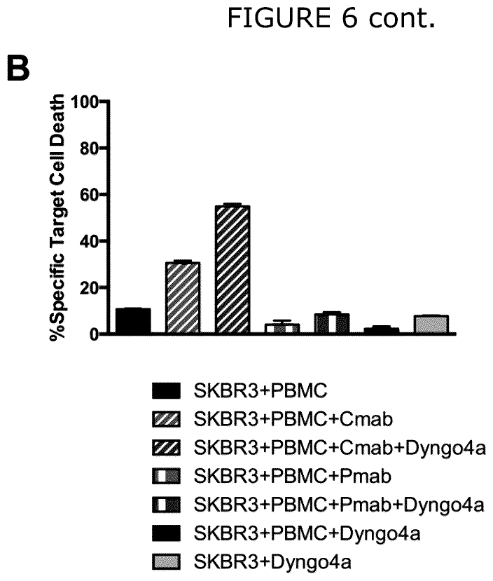

To better understand the dynamin inhibition-mediated increase in the sensitivities of breast cancer cell line to the mAb-mediated immune-dependent cytotoxicity, it was important to identify the PBMC cell subset which contributed to this effect in the in vitro assay. NK cells (Chung et al., 2014; Schnueriger et al., 2011), which express FcγRIII with high binding affinity for IgG1 were chosen to be examined in the context of mAb-mediated immune-dependent cytotoxicity assays by using CD56-depleted or enriched PBMCs a effector cells as opposed to unsorted PBMCs. It was revealed that NK cells are indeed the most likely cell subtype responsible for in vitro mAb mediated immune-dependent tumor cell cytotoxicity. Specific cell death of SKBR3 cells treated with Trastuzumab in the presence of a CD56-enriched fractionated PBMC population was increased in comparison to untreated controls, while SKBR3 cell death was negligible for both treated and untreated cells in the presence of CD56 depleted PBMCs (FIG. 6A). For untreated SKBR3 cells exposed to CD56+ PBMCs, specific cell death was increased above that of SKBR3 cells in the presence of a complete PBMC population and increased for cells treated with Trastuzumab to a comparable level to that of treated cells in the presence of a complete PBMC population. Moreover, a comparison of the efficacy of mAbs which bind to same antigen, and display distinct IgG domains (IgG1 and IgG2) was made. It was revealed that in vitro cytotoxicity against tumor cells occurred in the presence of IgG1 and was greatly enhanced following dynamin inhibition, but not IgG2, for which dynamin inhibition produced only a small increase in immune-effector-cell-mediated tumor cell death (FIG. 6B). Together these data suggest that the in vitro mAb mediated, immune-dependent tumor cytotoxicity observed throughout this study may have been FcγRIII dependent and NK cell mediated.

### Example 7

### Dynamin but not Clathrin Inhibition Improves ADCC in Response to Cetuximab

If only the plasma membrane levels of HER1 determine ADCC response then we would expect that any pharmacological blockade of HER1 internalization would be expected to enhance ADCC. Thus, numerous endocytosis inhibitors were tested to investigate this hypothesis. Importantly, it was found that inhibitors of clathrin coated vesicle (CCVs) formation, such as Pitstop2, caused HER1 to be trapped on the plasma membrane but did not increase ADCC in either Cetuximab-sensitive or resistant cell lines. However, when dynamin inhibitors such as chlorpromazine are used the present inventors observed clustering and recruitment of HER1 into CCVs that cannot detach from the plasma membrane.

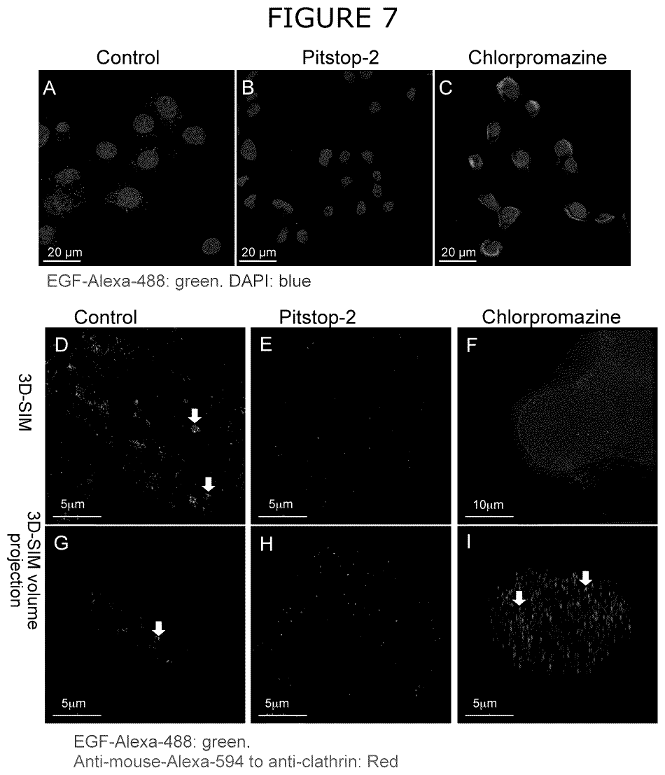

To discuss the experiments in more detail, A431 cells were incubated in serum-free medium with no drugs (control), or with Pitstop®-2 for 30 min or chlorpromazine for 30 min. During the last five minutes of treatment EGF-Alexa488 was added to monitor CME, after which cells were fixed and imaged (FIG. 7A-C). The control cells showed uptake of labeled EGF into early endosomal structures after 30 min, however both chlorpromazine and Pitstop®-2 showed no internalization, with EGF remaining bound to the cell surface (FIGS. 7B and C). Notably, abundant EGFR was trapped at the cell membrane in chlorpromazine-treated, but not Pitstop®-2-treated cells. Since the EGF was directly conjugated to Alexa⁴⁸⁸, without secondary antibody amplification, it was hypothesized that EGFRs are only observed when clustered into CCVs at the plasma membrane, but are not detectable as more diffuse binding to EGFR receptors on the cell surface that are not associated with CCVs. This would suggest that Pitstop®-2, but not chlorpromazine, inhibits CCV formation.

To directly test this proposal, the present inventors used super-resolution three dimensional structured illumination microscopy (3D-SIM) to compare the localization of clathrin with that of EGF in these cells. 3D-SIM provides an ˜8-fold improved volumetric resolution over conventional confocal laser scanning microscopy due to its lateral (x, y) resolution of 100-130 nm and axial (x, z) resolution of 250-300 nm (43). A single cell 3-D reconstruction is shown in FIG. 7D-F. In control cells, EGF-Alexa⁴⁸⁸ was internalized into endosomal structures (FIG. 7D) and this was inhibited by both Pitstop®-2 and chlorpromazine (FIGS. 7E and F). After either drug treatment, EGF was still evident at the plasma membrane (FIGS. 7E and F), supporting the conclusion that Pitstop®-2, but not chlorpromazine, inhibits CCV formation.

Volume projections of a small area of the cell periphery were used to examine the recruitment of clathrin and EGF-positive sites to the cell periphery (FIG. 7G-I). The angle of 3D projection was chosen to show the upper surface of the cell plasma membrane. Points of co-localisation between EGF-Alexa⁴⁸⁸ and Alexa⁵⁹⁴-conjugated antibodies (which detects clathrin) are rare in 3D-SIM due to the capture of frequency information differences between the 488 and 594 excitation/emission spectra. However, some sites of co-localization between EGF-Alexa⁴⁸⁸ and anti-clathrin-Alexa⁵⁹⁴ were observed (arrows in FIGS. 7G and I). A clathrin punctum was defined as being recruited an HER1-positive site when there was 50 nm or less distance between the clathrin and EGF puncta. Such close proximity of clathrin and EGF was evident in untreated control cells (FIGS. 7D and G), including rare yellow spots of complete overlap of the molecules. In Pitstop®-2-treated cells the EGF-Alexa⁴⁸⁸ was only observed at the plasma membrane but was rarely associated with clathrin (FIGS. 7E and H), suggesting that clathrin was not recruited the plasma membrane and consistent with the known Pitstop®-2 mechanism of action. In chlorpromazine-treated cells abundant clathrin puncta were observed within 100 nm of the plasma membrane. Many clathrin-positive puncta were within 50 nm of EGF puncta, including some sites of fluorescence colocalization. Therefore chlorpromazine does not inhibit clathrin recruitment to the plasma membrane. The increase in EGF clusters at the plasma membrane of chlorpromazine treated cells suggests that the CCVs remain associated with the plasma membrane after clathrin recruitment. The data show that chlorpromazine, but not Pitstop®-2, inhibits endocytosis at a point after clathrin recruitment and CCV formation.

Thus, the differential observed between the two very close steps of inhibition of CCV formation versus inhibition of CCV budding suggests that clustering of the plasma membrane HER1 is required for enhanced ADCC, not just increased plasma membrane levels. When a model is applied in which plasma membrane clustering and residency of the HER1 are required for ADCC then numerous outcomes in anti-HER1 based therapies can be explained.

### Materials and Methods for Examples 1-7

### Cell Lines

Cell lines used for Examples 1-7 were sourced from American Type Culture Collection (ATCC). In Table 2, the HER2/HER1 over-expression status of the cell lines is arbitrarily denoted using “+” based upon relative HER2 and HER1 expression level determined using ELISA.

TABLE 2

| | | HER2 | HER1 | |
|---|---|---|---|---|
| | | expression | expression | Therapeutic antibody |
| Cell line | Origin | status | status | Sensitive/Resistant |
| SKBR3 | Breast | +++ | + | Trastuzumab |
| | adenocarcinoma | | | Sensitive |
| BT474 | Breast ductal | ++ | | Trastuzumab |
| | carcinoma | | | Sensitive |
| HCC1569 | Breast metaplastic | ++ | | Trastuzumab |
| | carcinoma | | | Resistant |
| MDA-MB-468 | Breast | | +++ | Trastuzumab |
| | adenocarcinoma | | | Resistant |
| MDA-MB-231 | Breast | | ++ | Trastuzumab |
| | adenocarcinoma | | | Resistant |
| A431 | Squamous cell | | +++ | Cetuximab |
| | carcinoma | | | Sensitive |

### Tissue Culture

Cell lines were maintained at 37° C. with 5% CO2 in media supplemented with 10% (heat inactivated) fetal bovine serum (FBS) (Life Technologies, Gibco; Carlsbad, USA). The media in which each cell line was maintained were as follows; SKBR3 and SKBR3 derivatives were cultured in McCoy's medium (Life Technologies, Gibco), SKBR3 cells with acquired resistance to Trastuzumab were additionally cultured at all times with 10 μg/ml Trastuzumab (Roche, Genentech; San Francisco, USA). BT474, MCF7, MDA-MB-468 and MDA-MB-231 were cultured in Dulbecco's Modified Eagle Medium (DMEM) (Life Technologies, Gibco), containing L-glutamine (Life Technologies, Gibco), sodium pyruvate (Life Technologies, Gibco), and HEPES (Life Technologies, Gibco), MCF7 culture medium was additionally supplemented with 0.01 mg/ml insulin (Sigma-Aldrich; St. Louis, USA). HCC1569 cells were cultured in Roswell Park Memorial Institute (RPMI) 1640 media (Life Technologies, Gibco). Fresh human PBMCs were also maintained in RPMI 1640 (Life Technologies, Gibco) media.

### Cell Lysate Preparation

Cancer cells were grown in cell culture dishes to ˜80% confluence. Dishes were placed on ice and washed 3 times with PBS before addition of Rasio Immunoprecipitation Assay (RIPA) lysis buffer, (150 mM sodium chloride, 1.0% Triton X-100 (Sigma-Aldrich), 0.5% sodium deoxycholate, 0.1% Sodium Dodecyl Sulphate (SDS) (Sigma-Aldrich), 50 mM Tris (Sigma-Aldrich), at a pH of 8.0), supplemented with protease and phosphatase inhibitor cocktail (Sigma-Aldrich). Cells were scraped, and cell lysates were collected and stored at −80° C.

### ELISA Assays

HER2 total ELISAs were conducted in accordance with the manufacturer's instructions (Life Technologies, Invitrogen; Carlsbad, USA). 100 μL of recombinant-human-HER2 standards or 50 μg of test sample cell lysates were added into anti-HER2 antibody coated wells of 96 well plates in triplicate. Plates were incubated at room temperature for 2 hours. Solution was discarded from wells which were subsequently washed 4 times with washing solution. 100 μL of human-HER2 detection antibody solution was added to each well and plates were incubated at room temperature for 1 hr. Solution was discarded from wells followed by 4 washes with washing solution provided. 100 μL of anti-rabbit IgG HRP was added into each of the wells. Plates were covered and incubated at room temperature for 30 min. Solution was discarded and wells were again washed 4 times with washing solution provided. 100 μL of stabilized chromogen was added into each well and incubated at room temperature for 30 minutes followed by addition of 100 μL of stop solution into each well. Absorbance was measured at 450 nm. Optical densities of control blank wells were subtracted from test wells before optical densities were graphed.

HER1 total ELISAs were conducted in accordance with manufacturer's instructions (Merk, Millipore; Billerca, USA), 100 μL of recombinant human-HER1 standards and 50 μg of test sample lysates were added into anti-HER1 antibody-coated wells of a 96-well plate in triplicate. Plates were covered and stored at room temperature for 2 hr. Solution was discarded and plates were washed 4 times with washing buffer provided. 100 μL of detection antibody was added to each well before a 1 hr incubation at room temperature. Solution was discarded and wells were washed 4 times using wash buffer provided. 100 μL of anti-Rabbit IgG-HRP conjugate was added into each well and incubated at room temperature for 45 minutes. Wells were washed using wash buffer provided and 100 μL of 3,3′,5,′-Tetramethylbenzidine (TMB) substrate solution was added into each well and incubated in the dark for 45 min. 100 μL of stop solution was added to each well. Absorbance was measured at 450 nm. Optical densities of control wells were subtracted from test wells before optical densities were graphed.

### Immunofluorescence (IF) Assays

Cells were cultured as described above on coverslips until they reached ˜80% confluence. Coverslips were washed three times with PBS before a 20-min fixation with 4% Paraformaldehyde (PFA) (Sigma-Aldrich). Followed by 3 PBS washes. Subsequently, cells were permeabilized with 0.1% Triton X-100 (Sigma-Aldrich), and washed three times with PBS followed by a 10-min incubation in blocking solution (2% BSA/PBS) (Sigma-Aldrich). Coverslips were then immersed in primary antibody (Trastuzumab (Roche, Genentech) (0.42 μg/mL) and Cetuximab (Merk KGaA; Darmstadt, Germany) (0.25 μg/mL)) for 1 hr at room temperature. Following primary antibody incubation, coverslips were recovered and blocked 3 times with 10-min incubations with 2% BSA/PBS (Sigma-Aldrich). Coverslips were then immersed in secondary antibody (anti-human Alexa⁶⁴⁷ (Life Technologies, Invitrogen) (5 μg/mL) and anti-human Alexa⁵⁹⁴ (Life Technologies, Invitrogen) (5 μg/mL)) for 1 hr at room temperature. Coverslips were washed 3 times with 10-min incubations with 2% BSA/PBS (Sigma-Aldrich) followed by one 10-min incubation in PBS containing DAPI (Life Technologies, Invitrogen) (30 nM). Coverslips were submerged into water and mounted using Prolong-Gold anti-face (Life Technologies, Invitrogen) onto microscopy slides.

### Microscopy

Cells were analyzed using Olympus 510 META laser scanning confocal microscope (Zeiss) using a ×63 objective lens at excitation and emission wavelengths of 594 and 647 nm. Images were processed using inbuilt Zeiss software and figures were compiled using Adobe Photoshop CC 2014.2.0 and Adobe Illustrator CC (2014).

### Immune-Dependent Cytotoxicity Assays

Whole blood was collected from healthy human donors and diluted by half with 2% FBS/PBS (Life Technologies, Gibco). The blood was then layered onto 15 mL Lymphoprep (Stemcell Technologies; Vancouver, Canada) gradients, Next, samples were centrifuged at 800×g for 20-min in accordance with the manufacturer's instructions (Stemcell Technologies). The PBMC interface was isolated washed in 40 ml of 2% FBS/PBS (Life Technologies, Gibco) by centrifugation at 550×g for 15 minutes. The supernatants were discarded and PBMCs were resuspended into RPMI 1640 (Life Technologies, Gibco) media as previously described.

Tumor cells were cultured as described above. Cells were dissociated with 0.25% Trypsin EDTA (Life Technologies, Gibco) and resuspended into 10 ml of respective culture medium before centrifugation at 700 rpm for 5 min. The cell pellet was resuspended into 10 mL of PBS prior to centrifugation at 700 rpm for 5 min and then repeated. Cells were strained through and divided equally into four polystyrene FACS tubes: 1) unstained control, 2) 7-aminoactinomycin-D (7AAD) (BD Biosciences, BD Pharmingen; Franklin Lakes, USA) only control, 3) Carboxyfluorescein succinimidyl ester (CFSE) (Life Technologies, Invitrogen) only control and 4) CFSE test condition. CFSE (Life Technologies, Invitrogen) was added at a final concentration of 5 μM to CFSE (Life Technologies, Invitrogen) control and test tubes and incubated at room temperature for 10 min. An equal volume of 10% FBS/PBS (Life Technologies, Gibco) was added before the tubes were centrifuged at 1000 rpm for 5 minutes. The supernatant was removed from both tubes and the cells of the CFSE control tube were resuspended in 1 ml of 10% FBS/PBS (Life Technologies, Gibco) and the cells of the test condition were resuspended in 1 mL of respective medium.

PBMCs (Effector; E) and target tumor cells (Target; T) were counted separately. For assays in which an E:T ratio of 50:1 was used; 1.0×10⁶ PBMCs were combined with 2.0×10⁴ target cells in a final volume of 300 μL.

### Dynamin Inhibition Experiments

Trastuzumab (Roche, Genentech) or Cetuximab (Merk KGaA) was added to each of the conditions along side 2.0×10⁴ target cells at a final concentration of 60 μg/mL. 1.0×10⁶ effector cells were added to each tube and incubated at 37° C. for 3 hours. Dyngo4a (Abcam PLC, Abcam; Cambridge, USA) or chlorpromazine (Sigma-Aldrich, St Louis, CA) was added to respective conditions at a final concentration of 3 μM and 18 μM, respectively, before tubes were returned to 37° C. for an additional hour. Samples were treated as described above before analysis using BD FACS Canto Cell Analyzer.

### Super-Resolution Microscopy

A431 cells were seeded on coverslips in 12-well plates and incubated overnight to reach 80% confluence for super-resolution microscopy based on structured illumination. After a 3 hour-serum starvation the cells were treated with 30 μM of Pitstop®-2 or 18 μM chlorpromazine for 30 min, followed by 5 min uptake with EGF-Alexa488 (100 ng/ml). The uptake of EGF was stopped by washing three times in ice cold PBS. The cells were fixed in 4% paraformaldehyde/phosphate-buffered saline for 30 min and washed three times in phosphate-buffered saline before DAPI staining for 30 min. The coverslips were mounted on slides in Prolong Gold (Life technologies) for confocal analysis or glycerol-based imaging media for three dimensional structured illumination microscopy (3D-SIM). Confocal images were acquired using a Zeiss 510 Meta confocal with a 63 Å˜ objective. Reconstructing the subsequent images after modulating the illumination pattern produces super-resolution images with double the lateral and axial resolution and up to 10 μm into the cell. 3D-SIM images were captured on a Deltavision OMX V3 Imaging System (Applied Precision), EMCCD cameras (CascadeII 512×512 Photometrics) and using a 60×1.4-NA UPlanSApo oil-immersion objective (Olympus) with oil of a refractive index of 1.518. Each image log text file is available on request. Images were computationally reconstructed using Deltavision SoftWorX6.0 Beta 19 software (Applied Precision).

### CD56⁺ Isolation and Depletion Experiment

Approximately 4.0×10⁷ PBMCs were isolated as described and concentrated by centrifugation at 1000×g for 10 min and resuspended into medium as described above. PBMCs were stained with anti-human CD56 conjugated Allophycocyanin (APC) antibody (Affymetrix, eBiosciences; Santa Clara, USA) in accordance with the manufacturer's instructions (Affymetrix, eBiosciences). Stained PBMCs were then washed with 1 mL of 10% FBS/PBS (Life Technologies, Gibco) and centrifuged at 1000×g for 5 min, then the supernatant was removed and cells were washed twice more with 2% FBS/PBS (Life Technologies, Gibco) and PBS, respectively. Cells were sorted using Beckman and Coulter Moflo® Astrios cell sorter. Approximately 4.0×10⁷ unstained cells were maintained separately. For conditions containing unstained, unsorted PBMCs, 2.0×10⁴ target cells were treated with 60 μg/mL of Trastuzumab (Roche, Genentech) and combined with 1.0×10⁶ effector cells in a final volume of 300 μL. The same was conducted for conditions containing CD56-cell populations. For conditions containing CD56⁺ PBMC populations, 1.0×10⁴ target cells were combined with 60 μg/ml of Trastuzumab (Roche, Genentech) and 1.0×10⁵ effector cells into a final volume of 150 μL. Tubes were incubated at 37° C. for 4-hours. Samples were treated as described above before analysis using BD FACSCanto Cell Analyzer

### mAb Cell Surface Quantitation

Cells were dissociated as described previously and resuspended into ˜1 ml of respective medium. In triplicate, 1×10⁶ cells were treated with 60 μg/ml of either Trastuzumab (Roche, Genentech) or Cetuximab (Merk KGaA) and incubated at 37° C. with 5% CO2 for 4 hours. Dyngo4a (Abcam PLC, Abcam) was added to specified conditions following 3 hours of antibody incubation at a working concentration of 3 μM and incubated for an additional hour. Samples were removed from incubation and placed on ice before being washed three by centrifugation for 5 min at 1500 rpm. Following the final wash, cells were resuspended into 100 μL of 2% FBS/PBS (Life Technologies, Gibco) to which anti-human-Alexa⁶⁴⁷ (Life Technologies, Invitrogen) was added at a working concentration of 2 μg/mL then washed three additional times as described previously.

Following the final wash cells were fixed with 4% PFA (Sigma-Aldrich) for 20 min at room temperature. PFA (Sigma-Aldrich) was removed and cells were resuspended into 1 mL of PBS. Samples were analysed using BD FACSCanto. Measurements of geometric mean were used to calculate the mean fluorescence intensity of cell populations using FloJo 10.0.7 software.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Abd El-Rehim, D. M., S. E. Pinder, C. E. Paish, J. A. Bell, R. S. Rampaul, R. W. Blamey, J. F. Robertson, R. I. Nicholson, and I. O. Ellis. 2004. Expression and co-expression of the members of the epidermal growth factor receptor (EGFR) family in invasive breast carcinoma. British journal of cancer. 91:1532-1542.
Ahmad, S., S. Gupta, R. Kumar, G. C. Varshney, and G. P. Raghava. 2014, Herceptin resistance database for understanding mechanism of resistance in breast cancer patients. Scientific reports. 4:4483.
Barok, M., J. Isola, Z. Palyi-Krekk, P. Nagy, I. Juhasz, G. Vereb, P. Kauraniemi, A. Kapanen, M. Tanner, G. Vereb, and J. Szollosi. 2007. Trastuzumab causes antibody-dependent cellular cytotoxicity-mediated growth inhibition of submacroscopic JIMT-1 breast cancer xenografts despite intrinsic drug resistance. Molecular cancer therapeutics. 6:2065-2072.
Blick, S. K., and L. J. Scott. 2007. Cetuximab: a review of its use in squamous cell carcinoma of the head and neck and metastatic colorectal cancer. Drugs. 67:2585-2607.
Chung, S., Y. L. Lin, C. Reed, C. Ng, Z. J. Cheng, F. Malavasi, J. Yang, V. Quarmby, and A. Song. 2014. Characterization of in vitro antibody-dependent cell-mediated cytotoxicity activity of therapeutic antibodies—impact of effector cells. Journal of immunological methods. 407:63-75.
Clynes, R. A., T. L. Towers, L. G. Presta, and J. V. Ravetch. 2000. Inhibitory Fc receptors modulate in vivo cytotoxicity against tumor targets. Nature medicine. 6:443-446.
Di Lorenzo, G., G. Tortora, F. P. D'Armiento, G. De Rosa, S. Staibano, R. Autorino, M. D'Armiento, M. De Laurentiis, S. De Placido, G. Catalano, A. R. Bianco, and F. Ciardiello. 2002. Expression of epidermal growth factor receptor correlates with disease relapse and progression to androgen-independence in human prostate cancer. Clinical cancer research: an official journal of the American Association for Cancer Research. 8:3438-3444.
Eigenbrot, C., M. Ultsch, A. Dubnovitsky, L. Abrahmsen, and T. Hard. 2010. Structural basis for high-affinity HER2 receptor binding by an engineered protein. Proceedings of the National Academy of Sciences of the United States of America. 107:15039-15044.
Esendagli, G., K. Bruderek, T. Goldmann, A. Busche, D. Branscheid, E. Vollmer, and S. Brandau. 2008. Malignant and non-malignant lung tissue areas are differentially populated by natural killer cells and regulatory T cells in non-small cell lung cancer. Lung cancer (Amsterdam, Netherlands), 59:32-40.
Gajria, D., and S. Chandarlapaty. 2011. HER2-amplified breast cancer: mechanisms of trastuzumab resistance and novel targeted therapies. Expert review of anticancer therapy. 11:263-275.
Graus-Porta, D., R. R. Beerli, J. M. Daly, and N. E. Hynes. 1997. ErbB-2, the preferred heterodimerization partner of all ErbB receptors, is a mediator of lateral signaling. The EMBO journal. 16:1647-1655.
Hayashi, Y., Y. W. Widjono, K. Ohta, K. Hanioka, C. Obayashi, K. Itoh, Y. Imai, and H. Itoh. 1994. Expression of EGF, EGF-receptor, p53, v-erb B and ras p21 in colorectal neoplasms by immunostaining paraffin-embedded tissues. Pathology international. 44:124-130.
Hirsch, F. R., G. V. Scagliotti, C. J. Langer, M. Varella-Garcia, and W. A. Franklin. 2003. Epidermal growth factor family of receptors in preneoplasia and lung cancer: perspectives for targeted therapies. Lung cancer (Amsterdam, Netherlands). 41 Suppl 1:S29-42.
Izumi, Y., L. Xu, E. di Tomaso, D. Fukumura, and R. K. Jain. 2002. Tumor biology: herceptin acts as an anti-angiogenic cocktail. Nature. 416:279-280.
Kaufmann, R., P. Muller, G. Hildenbrand, M. Hausmann, and C. Cremer. 2011. Analysis of Her2/neu membrane protein clusters in different types of breast cancer cells using localization microscopy. Journal of microscopy. 242:46-54.
Klos, K. S., X, Zhou, S. Lee, L. Zhang, W. Yang, Y. Nagata, and D. Yu. 2003. Combined trastuzumab and paclitaxel treatment better inhibits ErbB-2-mediated angiogenesis in breast carcinoma through a more effective inhibition of Akt than either treatment alone. Cancer. 98:1377-1385.
Komarova, T. V., V. S. Kosorukov, O. Y. Frolova, I. V. Petrunia, K. A. Skrypnik, Y. Y. Gleba, and Y. L. Dorokhov. 2011. Plant-made trastuzumab (herceptin) inhibits HER2/Neu+ cell proliferation and retards tumor growth. PloS one. 6:e17541.
Mace, E. M., P. Dongre, H. T. Hsu, P. Sinha, A. M. James, S. S. Mann, L. R. Forbes, L. B. Watkin, and J. S. Orange. 2014. Cell biological steps and checkpoints in accessing NK cell cytotoxicity. Immunology and cell biology. 92:245-255.
Mccluskey, A., J. A. Daniel, G. Hadzic, N. Chau, E. L. Clayton, A. Mariana, A. Whiting, N. N. Gorgani, J. Lloyd, A. Quan, L. Moshkanbaryans, S. Krishnan, S. Perera, M. Chircop, L. von Kleist, A. B. McGeachie, M. T. Howes, R. G. Parton, M. Campbell, J. A. Sakoff, X. Wang, J. Y. Sun, M. J. Robertson, F. M. Deane, T. H. Nguyen, F. A. Meunier, M. A. Cousin, and P. J. Robinson. 2013. Building a better dynasore: the dyngo compounds potently inhibit dynamin and endocytosis. Traffic (Copenhagen, Denmark). 14:1272-1289.
Mellman, I., and Y. Yarden. 2013. Endocytosis and cancer. Cold Spring Harb Perspect Biol. 5(12):a016949
Mellstedt, H. 2003. Monoclonal antibodies in human cancer. Drugs Today (Barc). 39 Suppl C:1-16
Nahta, R., M. C. Hung, and F. J. Esteva. 2004. The HER-2-targeting antibodies trastuzumab and pertuzumab synergistically inhibit the survival of breast cancer cells. Cancer research. 64:2343-2346.
Narayan, M., J. A. Wilken, L. N. Harris, A. T. Baron, K. D. Kimbler, and N. J. Maihle. 2009. Trastuzumab-induced HER reprogramming in “resistant” breast carcinoma cells. Cancer research. 69:2191-2194.
Noguchi T, Ritter G, and H. Nishikawa. 2013. Antibody-based therapy in colorectal cancer. Immunotherapy. 5(5): 533-45
Olayioye, M. A. 2001. Update on HER-2 as a target for cancer therapy: intracellular signaling pathways of ErbB2/HER-2 and family members. Breast cancer research: BCR. 3:385-389.
Ooi, A., T. Takehana, X. Li, S. Suzuki, K. Kunitomo, H. Iino, H. Fujii, Y. Takeda, and Y. Dobashi. 2004. Protein overexpression and gene amplification of HER-2 and EGFR in colorectal cancers: an Immunohistochemical and fluorescent in situ hybridization study. Modern pathology: an official journal of the United States and Canadian Academy of Pathology, Inc. 17:895-904.
Patel, D., X. Guo, S. Ng, M. Melchior, P. Balderes, D. Burtrum, K. Persaud, X. Luna, D. L. Ludwig, and X. Kang. 2010. IgG isotype, glycosylation, and EGFR expression determine the induction of antibody-dependent cellular cytotoxicity in vitro by cetuximab. Human antibodies. 19:89-99.
Piccart-Gebhart, M. J., M. Procter, B. Leyland-Jones, A. Goldhirsch, M. Untch, I. Smith, L. Gianni, J. Baselga, R. Bell, C. Jackisch, D. Cameron, M. Dowsett, C. H. Barrios, G. Steger, C.-S. Huang, M. Andersson, M. Inbar, M. Lichinitser, I. Lang, U. Nitz, H. Iwata, C. Thomssen, C. Lohrisch, T. M. Suter, J. Rüschoff, T. Sütő, V. Greatorex, C. Ward, C. Straehle, E. McFadden, M. S. Dolci, and R. D. Gelber. 2005. Trastuzumab after Adjuvant Chemotherapy in HER2-Positive Breast Cancer. New England Journal of Medicine. 353:1659-1672.
Pinkas-Kramarski, R., M. Shelly, B. C. Guarino, L. M. Wang, L. Lyass, I. Alroy, M. Alimandi, A. Kuo, J. D. Moyer, S. Lavi, M. Eisenstein, B. J. Ratzkin, R. Seger, S. S. Bacus, J. H. Pierce, G. C. Andrews, and Y. Yarden. 1998. ErbB tyrosine kinases and the two neuregulin families constitute a ligand-receptor network. Molecular and cellular biology. 18:6090-6101.
Porebska, I., A. Harlozinska, and T. Bojarowski. 2000. Expression of the tyrosine kinase activity growth factor receptors (EGFR, ERB B2, ERB B3) in colorectal adenocarcinomas and adenomas. Tumor biology: the journal of the International Society for Oncodevelopmental Biology and Medicine. 21:105-115.
Pueyo, G., R. Mesia, A. Figueras, A. Lozano, M. Baro, S. Vazquez, G. Capella, and J. Balart. 2010. Cetuximab may inhibit tumor growth and angiogenesis induced by ionizing radiation: a preclinical rationale for maintenance treatment after radiotherapy. The oncologist. 15:976-986.
Scaltriti, M., C. Verma, M. Guzman, J. Jimenez, J. L. Parra, K. Pedersen, D. J. Smith, S. Landolfi, S. Ramon y Cajal, J. Arribas, and J. Baselga. 2009. Lapatinib, a HER2 tyrosine kinase inhibitor, induces stabilization and accumulation of HER2 and potentiates trastuzumab-dependent cell cytotoxicity. Oncogene. 28:803-814.
Schnueriger, A., R. Grau, P. Sondermann, T. Schreitmueller, S. Marti, and M. Zocher. 2011. Development of a quantitative, cell-line based assay to measure ADCC activity mediated by therapeutic antibodies. Molecular immunology. 48:1512-1517.
Scott, A. M., J. D. Wolchok, and L. J. Old. 2012. Antibody therapy of cancer. Nature reviews. Cancer. 12:278-287.
Srivastava, R. M., S. C. Lee, P. A. Andrade Filho, C. A. Lord, H. B. Jie, H. C. Davidson, A. Lopez-Albaitero, S. P. Gibson, W. E. Gooding, S. Ferrone, and R. L. Ferris. 2013. Cetuximab-activated natural killer and dendritic cells collaborate to trigger tumor antigen-specific T-cell immunity in head and neck cancer patients. Clinical cancer research: an official journal of the American Association for Cancer Research. 19:1858-1872.
Suo, Z., B. Risberg, M. G. Kalsson, K. Willman, A. Tierens, E. Skovlund, and J. M. Nesland. 2002. EGFR family expression in breast carcinomas. c-erbB-2 and c-erbB-4 receptors have different effects on survival. The Journal of pathology. 196:17-25.
Tzahar, E., H. Waterman, X. Chen, G. Levkowitz, D. Karunagaran, S. Lavi, B. J. Ratzkin, and Y. Yarden. 1996. A hierarchical network of interreceptor interactions determines signal transduction by Neu differentiation factor/neuregulin and epidermal growth factor. Molecular and cellular biology. 16:5276-5287.
Vincenzi, B., D. Santini, A. Russo, M. Silletta, M. Gavasci, F. Battistoni, G. Di Cuonzo, L. Rocci, N. Gebbia, and G. Tonini. 2006. Angiogenesis modifications related with cetuximab plus irinotecan as anticancer treatment in advanced colorectal cancer patients. Annals of oncology: official journal of the European Society for Medical Oncology/ESMO. 17:835-841.
Vu, T., and F. X. Claret. 2012. Trastuzumab: updated mechanisms of action and resistance in breast cancer. Frontiers in oncology. 2:62.
Wieduwilt, M. J., and M. M. Moasser. 2008. The epidermal growth factor receptor family: biology driving targeted therapeutics. Cellular and molecular life sciences: CMLS. 65:1566-1584.
Witton, C. J., J. R. Reeves, J. J. Going, T. G. Cooke, and J. M. Bartlett. 2003. Expression of the HER1-4 family of receptor tyrosine kinases in breast cancer. The Journal of pathology. 200:290-297.
Wolff, A. C., M. E. Hammond, J. N. Schwartz, K. L. Hagerty, D. C. Allred, R. J. Cote, M. Dowsett, P. L. Fitzgibbons, W. M. Hanna, A. Langer, L. M. McShane, S. Paik, M. D. Pegram, E. A. Perez, M. F. Press, A. Rhodes, C. Sturgeon, S. E. Taube, R. Tubbs, G. H. Vance, M. van de Vijver, T. M. Wheeler, and D. F. Hayes. 2007. American Society of Clinical Oncology/College of American Pathologists guideline recommendations for human epidermal growth factor receptor 2 testing in breast cancer. Archives of pathology & laboratory medicine. 131:18-43.
Yarden, Y. 2001. Biology of HER2 and its importance in breast cancer. Oncology. 61 Suppl 2:1-13.
Zimmer, J., H. Bausinger, E. Andres, L. Donato, D. Hanau, F. Hentges, A. Moretta, and H. de la Salle. 2007. Phenotypic studies of natural killer cell subsets in human transporter associated with antigen processing deficiency. PloS one. 2:e1033.

1-33. (canceled)

34. A method for treating a subject with a cell surface receptor element-positive tumor, the method comprising:
stratifying the subject into a treatment subgroup selected from responder and non-responder to a therapeutic agent that binds the cell surface receptor, wherein the subject is stratified by determining the clustering status of the cell surface receptor element of a tumor sample obtained from the subject
and administering the therapeutic agent that binds to the cell-surface receptor to the subject on the basis that the subject is stratified into a responder treatment subgroup or administering a cancer therapy other than the therapeutic agent that binds to the cell-surface receptor to the subject on the basis that the subject is stratified into the non-responder treatment subgroup, wherein the subject is stratified into the responder treatment subgroup when at least 40% of the cell surface receptor elements on a tumor cell of the tumor sample obtained from the patient are present in clusters, and the subject is stratified into the non-responder treatment subgroup when less than 40% of the cell surface receptor elements on a tumor cell of the tumor sample obtained from the patient are present in clusters.

35-36. (canceled)

37. The method according to claim 34, wherein the therapeutic agent is an antibody.

38-41. (canceled)

42. A method according to claim 34, further comprising contacting the tumor sample obtained from the subject with a ligand of the cell surface receptor

43. The method according to claim 42, further comprising measuring an antibody-dependent cell-mediated cytotoxicity (ADCC) activity or a complement-mediated cytotoxicity (CDC) activity of the ligand, wherein the subject is stratified into the responder treatment subgroup when the level of clustering of the cell surface receptor elements on the tumor cell of the tumor sample corresponds to the level of clustering of the cell surface receptor elements on a therapeutic agent responsive control tumor cell and when the ADCC or CDC activity of the ligand on the sample cell surface receptor element positive tumor cell is at least 30% of the ADCC or CDC activity of the ligand on the therapeutic agent responsive control tumor cell.

44. The method according to claim 34, wherein the cell surface receptor elements are selected from growth factor receptors, cytokine receptors, hormone receptors, tumor differentiation antigens and cluster of differentiation molecules.

45. The method according to claim 44, wherein the growth factor receptor is selected from: epidermal growth factor receptor family members, insulin receptor, insulin-like growth factor receptor, vascular endothelial growth factor receptor, tumor necrosis factor receptor, fibroblast growth factor receptor, hepatocyte growth factor receptor, platelet derived growth factor receptor, and ephrin receptor family members.

46. The method according to claim 44, wherein the cytokine receptor is selected from the group consisting of: cytokine receptor common gamma chain, interleukin-10 receptor alpha chain, interleukin-10 receptor beta chain, interleukin-12 receptor beta-1 chain, interleukin-12 receptor beta-2 chain, interleukin-13 receptor alpha-1 chain, interleukin-13 receptor alpha-2 chain, interleukin-17 receptor, interleukin-17b receptor, interleukin 21 receptor precursor, interleukin-1 receptor, type I, interleukin-1 receptor, type II, interleukin-2 receptor alpha chain, interleukin-2 receptor beta chain, interleukin-3 receptor alpha chain, interleukin-4 receptor alpha chain, interleukin-5 receptor alpha chain, interleukin-6 receptor alpha chain, interleukin-6 receptor beta chain, interleukin-7 receptor alpha chain, high affinity interleukin-8 receptor a, high affinity interleukin-8 receptor b, interleukin-9 receptor, interleukin-18 receptor, TNF-related apoptosis-inducing ligand, toll-like receptor 1, toll-like receptor, toll-like receptor, cx3c chemokine receptor 1, C-X-C chemokine receptor type 3, C-X-C chemokine receptor type 4, C-X-C chemokine receptor type 5, C-X-C chemokine receptor type 6, chemokine binding protein 2, C-C chemokine receptor type 1, C-C chemokine receptor type 2, C-C chemokine receptor type 3, C-C chemokine receptor type 4, C-C chemokine receptor type 5, C-C chemokine receptor type 6, C-C chemokine receptor type 8, C-C chemokine receptor type, C-C chemokine receptor type 10, C-C chemokine receptor type 11, chemokine receptor-like 1, chemokine receptor-like 2, and chemokine XC receptor 1. In specific embodiments, the cell surface receptor is an epidermal growth factor receptor family member (e.g., EGFR (HER1), ErbB2 (HER2), ErbB3, ErbB4).

47. The method according to claim 44, wherein the tumor differentiation antigen is selected from the group consisting of: α-fetoprotein (AFP), carcinoembryonic antigen (CEA), CA-125, mucins, epithelial tumor antigen (ETA), colon-specific antigen-p (CSA-p), tyrosinase, prostate-specific membrane antigen (PSMA), A33-antigen, transferrin receptor, tenascin, CA-IX and melanoma associated antigen.

48. The method according to claim 44, wherein the hormone receptor is selected from the group consisting of estrogen receptor and progesterone receptor.

49. The method according to claim 34, wherein the cell surface receptor elements are selected from MAC-1, opioid receptors, FC receptors, serotonin receptors, β-adrenergic receptors, leptin receptor, statin receptors, FAS receptor, BAFF receptor, FLT3 receptor and fibronectin receptor.

50. The method according to claim 43, wherein the cell surface receptor elements are selected from members of the HER family.
